(19) United States
(12) Patent Application Publication
(10) Pub. No.: US 2016/0270757 A1
(43) Pub. Date: Sep. 22, 2016

(54) IMAGE-PROCESSING APPARATUS, IMAGE-PROCESSING METHOD, AND PROGRAM

(71) Applicant: KONICA MINOLTA, INC., Tokyo (JP)

(72) Inventors: Tadamasa TOMA, Kadoma-shi (JP); Jun OHMIYA, Kyotanabe-shi (JP); Bumpei TOJI, Hashima-shi (JP)

(73) Assignee: KONICA MINOLTA, INC., Tokyo (JP)

(21) Appl. No.: 14/442,281

(22) PCT Filed: Nov. 11, 2013
(86) PCT No.: PCT/JP2013/006625

§ 371 Date: May 12, 2015

(30) Foreign Application Priority Data
Nov. 15, 2012 (JP) 2012-251583

(51) Int. Cl. A61B 8/08; A61B 8/00

(52) U.S. Cl. CPC A61B 8/0841; A61B 8/463; A61B 8/0891; A61B 8/5207; A61B 8/483; A61B 8/466; A61B 8/5223; A61B 8/5246; A61B 8/4483

(57) ABSTRACT

An image processing apparatus capable of displaying an image for providing guidance in moving an instrument to a target part in a subject to a user with high visual perceptibility. The image processing apparatus may be an ultrasound diagnostic apparatus including: a three-dimensional image analyzer determining target position indicating a three-dimensional position of the target part based on a three-dimensional image including the target part; a position information acquirer acquiring instrument position indicating a three-dimensional position of the instrument; a display state determiner selecting one display state from at least two display states, based on a positional relationship between the target part and the instrument; an assist image generator generating an assist image for the selected display state by using the target position and the instrument position; and a display controller performing control for outputting the assist image generated by the assist image generation unit to a display device.

### TECHNICAL FIELD

The present invention relates to an image processing apparatus and an image processing method for generating an image for providing guidance in moving an instrument to a target part of a subject.

### BACKGROUND ART

Image diagnostic apparatuses for a living body, such as X-ray diagnostic apparatuses, MR (magnetic resonance) diagnostic apparatuses, and ultrasound diagnostic apparatuses, have widely spread. Particularly, ultrasound diagnostic apparatuses have advantages such as noninvasiveness and real-time performance, and are widely used for diagnosis and medical checkup. Ultrasound diagnostic apparatuses are used for diagnosis of a wide variety of body parts, such as the heart, blood vessels, the liver, and the breasts. In recent years, attention is being given to diagnosis of blood vessels, such as the carotid artery, for assessing the risk of arterial sclerosis. However, since vascular diagnosis requires much skill. Accordingly, ultrasound diagnostic apparatuses displaying images providing guidance to examiners are being proposed. One example of such an ultrasound diagnostic device is described in Patent Document 1.

Further, in recent years, intra-surgery navigation systems displaying the positional relationship between a part of a patient body and a surgical instrument during surgery are being proposed. Such intra-surgery navigation systems are used, for example, in order to improve visual perceptibility of where a tumor or a blood vessel is located, and to improve surgical safety through display of a position of a surgical instrument with respect to a part of the patient body that is the surgical target, such as a bone or an organ.

### CITATION LIST

### Patent Literature

### [Patent Literature 1]

Japanese Patent Application Publication No. 2010-051817

### SUMMARY OF INVENTION

### Technical Problem

However, ultrasound diagnostic apparatuses and intra-surgery navigation systems as described above pose a problem that images displayed to users, who may be examiners and operators, do not have high visual perceptibility.

The present invention, therefore, provides an image processing apparatus that is capable of displaying, to a user with high visual perceptibility, an image for providing guidance in moving an instrument to a target part of a subject.

### Solution to Problem

One aspect of the present invention is an image processing apparatus for generating an assist image that is an image providing guidance in moving an instrument to a target part of a subject, including: a three-dimensional image analyzer determining, as target position information, a three-dimensional position of the target part based on a three-dimensional image including the target part; a position information acquirer acquiring instrument position information indicating a three-dimensional position of the instrument; a display state determiner selecting one display state from at least two display states based on a positional relationship between the target part and the instrument; an assist image generator generating an assist image for the selected display state by using the target position information and the instrument position information; and a display controller performing control for outputting the assist image generated by the assist image generator to a display device.

Such aspects of the present invention, including those that are general and those that are specific, may be realized by a system, a method, an integrated circuit, a computer program, or a computer-readable recording medium such as a CD-ROM, or may be realized by any combination of a system, a method, an integrated circuit, a computer program, or a computer-readable recording medium such as a CD-ROM.

### Advantageous Effects of Invention

The present invention enables displaying, to a user with high visual perceptibility, an image for providing guidance in moving an instrument to a target part of a subject.

### BRIEF DESCRIPTION OF DRAWINGS

FIG. 1A is a schematic diagram illustrating probe and scan plane.

FIG. 1B is a diagram illustrating two scanning directions when scanning carotid artery with probe.

FIG. 1C is a diagram illustrating one example of ultrasound image acquired by long-axis scan.

FIG. 1D is a diagram illustrating one example of ultrasound image acquired by short-axis scan.

FIG. 2A is a cross-sectional view illustrating structure of arterial vessel in short-axis cross-section.

FIG. 2B is a cross-sectional view illustrating structure of arterial vessel in long-axis cross-section.

FIG. 2C is a cross-sectional view illustrating boundary between tunica intima and tunica adventitia in short-axis cross-section.

FIG. 2D is a cross-sectional view illustrating one example of hypertrophy of intima-media complex in long-axis cross-section.

FIG. 3 is a block diagram illustrating structure of ultrasound diagnostic apparatus according to assumed technology.

FIG. 4 is a flowchart illustrating operation of ultrasound diagnostic apparatus according to assumed technology.

FIG. 5 is a diagram illustrating example of screen structure including assist image and live image.

FIG. 6 is block diagram illustrating structure of ultrasound diagnostic apparatus according to first embodiment.

FIG. 7 is a flowchart illustrating operation of ultrasound diagnostic apparatus according to first embodiment.

FIG. 8 A is a diagram illustrating example flow of three-dimensional image generation.

FIG. 8B is a diagram illustrating example flow of three-dimensional image generation.

FIG. 8C is a diagram illustrating example flow of three-dimensional image generation.

FIG. 8D is a diagram illustrating example flow of three-dimensional image generation.

FIG. 9A is a diagram illustrating position and orientation of measurement target in three-dimensional image.

FIG. 9B is a diagram illustrating position of measurement target in long-axis cross-section.

FIG. 9C is a diagram illustrating position of measurement target in short-axis cross-section.

FIG. 10 is a flowchart illustrating one example of operation of switching screen display.

FIG. 11A is a diagram illustrating one example of carotid artery (measurement target) in three-dimensional space.

FIG. 11B is a diagram illustrating one example of second display state.

FIG. 11C is a diagram illustrating one example of first display state.

FIG. 12 is a flowchart illustrating one example of operation where hysteresis is applied to switching of screen display.

FIG. 13A is a diagram illustrating one example of carotid artery (measurement target) in three-dimensional space.

FIG. 13B is a diagram illustrating one example of carotid artery in long-axis direction in three-dimensional space.

FIG. 13C is a diagram illustrating one example of carotid artery in short-axis direction in three-dimensional space.

FIG. 13D is a diagram illustrating one example of display after switching including combination of live image in long-axis direction and assist image in short-axis direction.

FIG. 14A is a diagram illustrating one example of assist image before switching, with viewpoint in long-axis direction.

FIG. 14B is a diagram illustrating one example of assist image after switching, with viewpoint in short-axis direction.

FIG. 15A is a diagram illustrating one example of assist image before switching, with viewpoint in long-axis direction.

FIG. 15B is a diagram illustrating assist image after switching, with viewpoint in short-axis direction and increased magnification ratio.

FIG. 16 is a flowchart illustrating one example of operation for switching assist image settings.

FIG. 17A is a diagram illustrating another example of second display state.

FIG. 17B is a diagram illustrating another example of first display state.

FIG. 18 is a flowchart illustrating operation of ultrasound diagnostic apparatus according to second embodiment.

FIG. 19A is a diagram illustrating system for acquiring position information of probe with camera.

FIG. 19B is a diagram illustrating specific example 1 where position information of probe is not acquired.

FIG. 19C is a diagram illustrating specific example 1 of screen displaying warning information.

FIG. 19D is a diagram illustrating specific example 2 where position information of probe is not acquired.

FIG. 19E is a diagram illustrating specific example 2 of screen displaying warning information.

FIG. 20A is a diagram illustrating display example 1 where subject posture and orientation of three-dimensional image are associated with each other.

FIG. 20B is a diagram illustrating display example 2 where subject posture and orientation of three-dimensional image are associated with each other.

FIG. 21 is a diagram illustrating example of screen structured by using assist image including images from two viewpoints.

FIG. 22 is a flowchart illustrating operation of ultrasound diagnostic apparatus according to third embodiment.

FIG. 23 is a schematic diagram illustrating example of installation of intra-surgery navigation system.

FIG. 24 is a diagram illustrating outline of how information is imported into virtual three-dimensional space.

FIG. 25 is a block diagram illustrating structure of image processing apparatus according to fourth embodiment.

FIG. 26 is a flowchart illustrating operation of image processing apparatus according to fourth embodiment.

FIG. 27A is a diagram illustrating one example of assist image for second display state.

FIG. 27B is a diagram illustrating one example of assist image for first display state.

FIG. 28A is a diagram illustrating example of physical format of flexible disk (main body of recording medium).

FIG. 28B is a diagram illustrating front-side appearance of flexible disk, cross-sectional structure of flexible disk, and flexible disk.

FIG. 28C is a diagram illustrating structure for recording/reproducing program in/from flexible disk.

### DESCRIPTION OF EMBODIMENTS

### (Knowledge Forming Basis of Present Invention)

The inventors of the present invention found that the following problems arise in image processing apparatuses, such as the ultrasound diagnostic apparatuses and the intra-surgery navigation systems described in the “Background Art” section of the present disclosure.

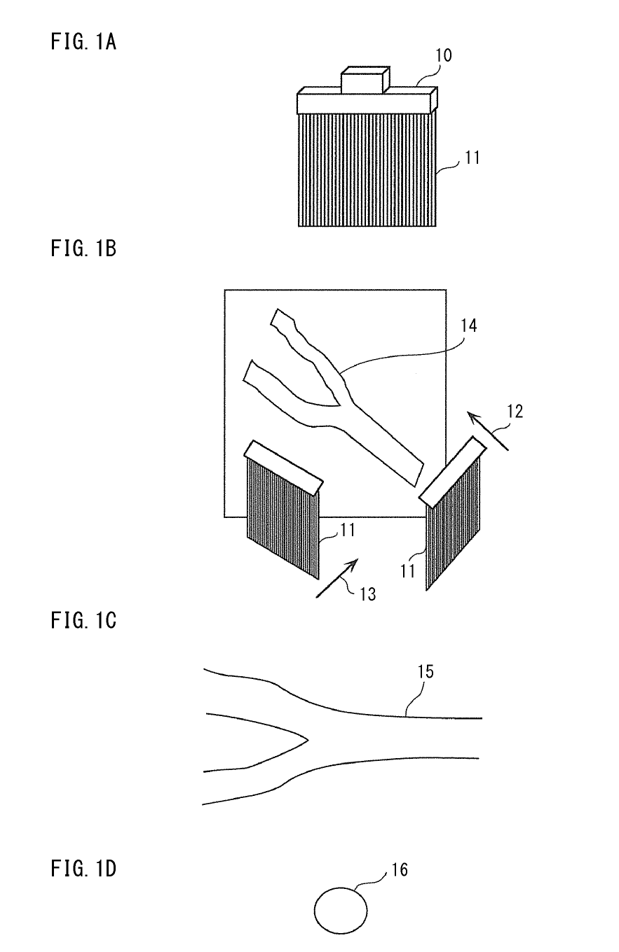

First, description will be given of ultrasound diagnosis of the carotid artery. FIGS. 1A to 1D each illustrate a carotid artery image obtained by an ultrasound scan. FIG. 1A schematically illustrates a probe and a scan plane. FIG. 1B illustrates two scanning directions when scanning the carotid artery with the probe. FIG. 1C illustrates one example of an ultrasound image acquired by a long-axis scan. FIG. 1D illustrates one example of an ultrasound image acquired by a short-axis scan.

The probe 10 includes ultrasound transducers (not illustrated). For example, when the ultrasound transducers are one-dimensionally arranged, an ultrasound image is obtained with respect to a two-dimensional scan plane 11 immediately below the ultrasound transducers, as illustrated in FIG. 1A. In the diagnosis of the carotid artery, typically, images in two directions are acquired. One direction is a direction 12 (short-axis direction) in which the carotid artery 14 is cut into round slices, and the other is a direction 13 (long-axis direction) that is substantially orthogonal to the short-axis direction 12, as illustrated in FIG. 1B. When scanning the carotid artery 14 with the probe 10 in the long-axis direction 13, a long-axis directional vascular image as illustrated in FIG. 1C is acquired. When scanning the carotid artery 14 with the probe 10 in the short-axis direction 12, a short-axis directional vascular image as illustrated in FIG. 1D is acquired.

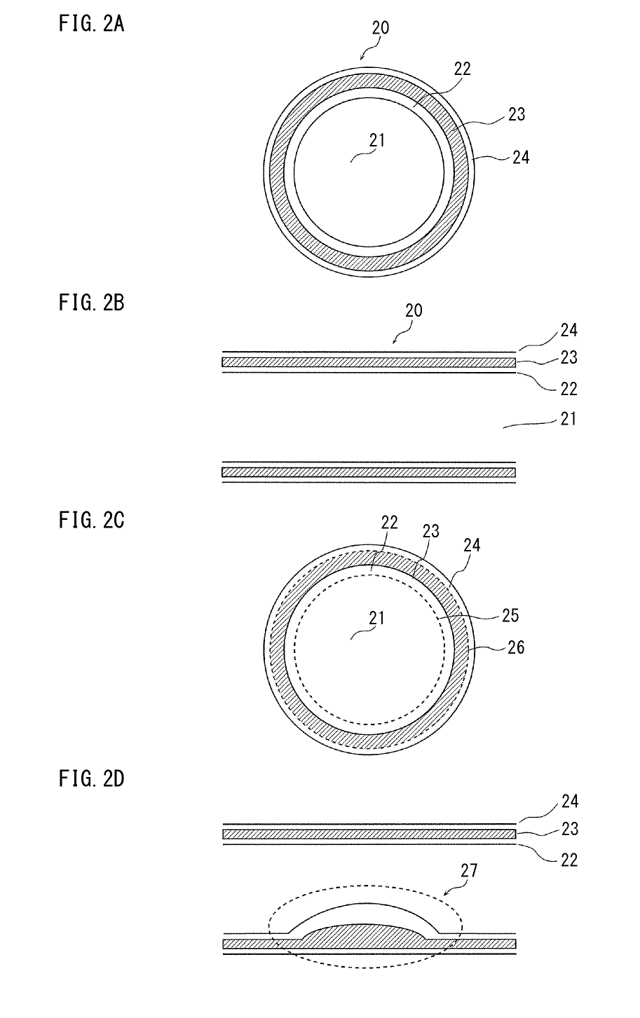

With reference to FIGS. 2A to 2D, next, description will be given of the structure of a vascular wall in an artery, for the following reason. In the diagnosis of the carotid artery, the progress of arterial sclerosis is grasped by using the thickness of a vascular wall as an index. FIG. 2A is a sectional view illustrating the structure of an arterial vessel in a short-axis cross-section. FIG. 2B is a sectional view illustrating the structure of an arterial vessel in a long-axis cross-section. FIG. 2C is a sectional view illustrating a boundary between the tunica intima and the tunica adventitia in a short-axis cross-section. FIG. 2D is a sectional view illustrating one example of the hypertrophy of the intima-media complex in a long-axis cross-section.

As illustrated in FIGS. 2A and 2B, a vascular wall 20 of the artery includes three layers, namely, the tunica intima 22, the tunica media 23, and the tunica adventitia 24. As illustrated FIG. 2C and FIG. 2D, the progress of arterial sclerosis causes hypertrophy of mainly the tunica intima 22 and the tunica media 23. In the ultrasound diagnosis of the carotid artery, accordingly, the thickness of the intima-media complex composed of the tunica intima 22 and the tunica media 23 is measured by detecting a lumen-intima boundary 25 and a media-adventitia boundary 26, which are illustrated in FIG. 2C. A portion of the intima-media complex whose thickness exceeds a certain value is called a plaque 27. A plaque causes a structural change in the vascular wall as illustrated in the long-axis image of FIG. 2D. Typically, both the short-axis image and the long-axis image are checked for examining the plaque 27.

Treatment such as medication or surgical separation of the plaque 27 is required depending on the thickness, the size, etc., of the plaque 27. Therefore, correctly measuring the thickness of the intima-media complex becomes a key in the diagnosis. However, the thickness of the intima-media complex changes depending on the region that is measured. Further, an examiner cannot easily grasp three-dimensionally the shape of the carotid artery, which runs inside the neck. Therefore, diagnosis of the carotid artery requires skill and experience. Further, when medicinal treatment is applied, a specific position of the plaque 27 is measured periodically in order to confirm the effect of the treatment. That is, a diagnosis is made of whether the thickness, the area, the volume, etc., of the plaque 27 are being effectively reduced by the treatment. Here, it is important that the plaque 27 be measured at the same position and in the same orientation each time. This measurement requires skill and experience.

Hence, an ultrasound diagnostic apparatus 30 is proposed that provides guidance to an examiner by displaying an ultrasound live image (i.e., a real-time ultrasound image acquired by a probe) and in addition, how the probe is to be moved in order to acquire an ultrasound image in a position and orientation that are to be measured.

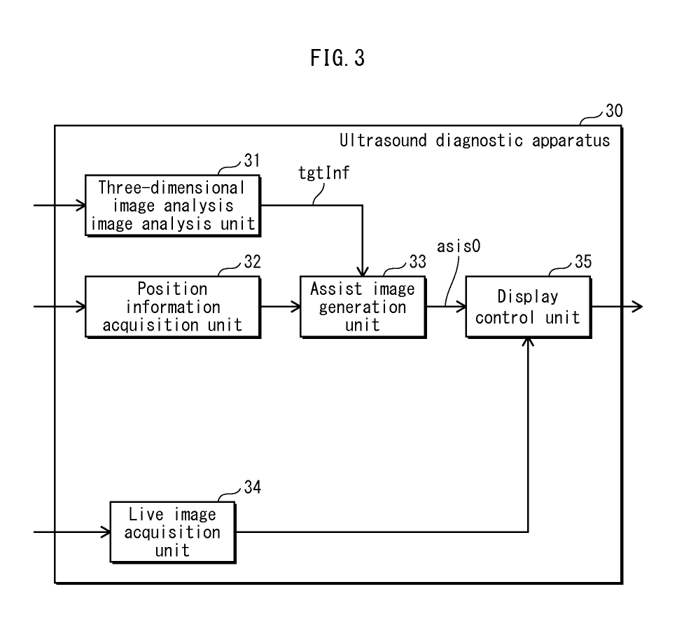

FIG. 3 is a block diagram illustrating the structure of the ultrasound diagnostic apparatus 30.

As illustrated in FIG. 3, the ultrasound diagnostic apparatus 30 includes a three-dimensional image analysis unit 31, a position information acquisition unit 32, an assist image generation unit 33, a live image acquisition unit 34, and a display control unit 35.

The three-dimensional image analysis unit 31 analyzes a three-dimensional image (hereinafter, referred to as a 3D image) acquired in advance. Further, the three-dimensional image analysis unit 31 determines target position information tgtInf including a three-dimensional position (hereinafter, also simply referred to as a position) and an orientation of a measurement target part of a subject (hereinafter, also referred to as a measurement target). Further, the three-dimensional image analysis unit 31 outputs the target position information tgtInf so determined to the assist image generation unit 33.

The position information acquisition unit 32 acquires instrument position information indicating a current scan position and a current orientation of the probe 10, by use of, for example, a magnetic sensor or an optical camera.

The assist image generation unit 33 generates an assist image asis0, in which the measurement plane of the measurement target and information concerning the position and the orientation of the current scan plane are superimposed on the 3D image, based on the 3D image, the target position information tgtInf, and the instrument position information.

The display control unit 35 causes a display device 150 to display the assist image, along with a live image (ultrasound image) at the current scan position.

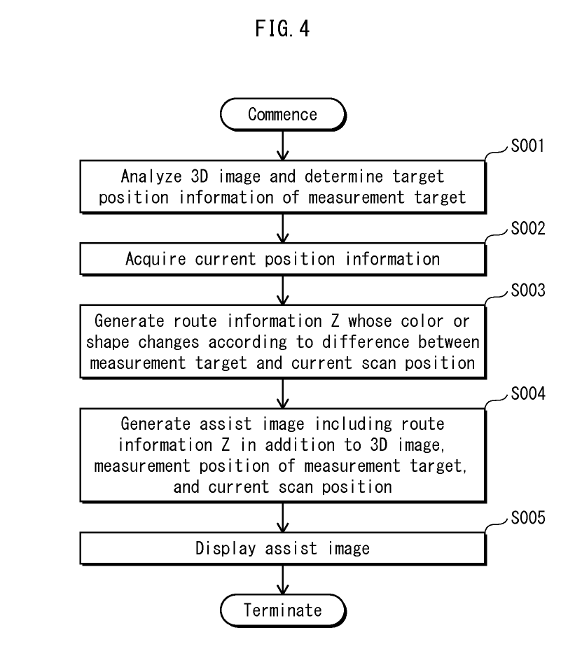

FIG. 4 is a flowchart illustrating the operation of the ultrasound diagnostic apparatus 30. It is assumed herein that a 3D image showing the shape of the diagnosis-target organ has been generated in advance.

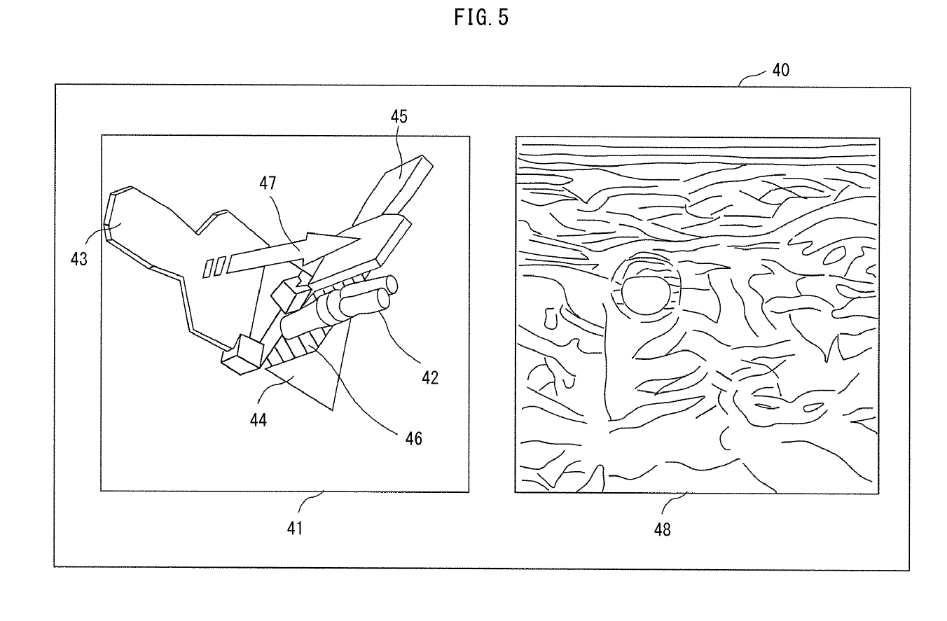

First, the three-dimensional image analysis unit 31 analyzes the 3D image to determine the target position information including the position and the orientation of the measurement target (Step S001). Next, the position information acquisition unit 32 acquires the instrument position information indicating the current scan position and the current orientation of the probe 10 (Step S002). Next, the assist image generation unit 33 calculates a difference between the position of the measurement target and the current scan position to generate route information Z for changing the color or the shape of the image to be displayed in accordance with the difference (Step S003). Then the assist image generation unit 33 generates an assist image containing the route information Z in addition to the 3D image, the position of the measurement target, and the current scan position (Step S004). The display control unit 35 causes the display device 150 to display a screen 40 obtained by combining an assist image 41 with a live image 48 (ultrasound image) at the current scan position, as illustrated in, for example, FIG. 5 (Step S005). The assist image 41 includes a 3D image 42 showing the shape of the organ including the target part, an image 43 showing the current position of the probe 10, an image 44 showing the current scan plane, an image 46 showing the scan plane of the measurement target, an image 45 showing the position in which the probe 10 is to be moved for scanning the measurement target, and an arrow 47 indicating the direction in which the probe 10 is to be moved.

Typically, an examiner positions the scan plane at the measurement target while moving the probe by first performing rough alignment, and then performing fine adjustment. The examiner mainly refers to the assist image when performing the rough alignment, and mainly refers to the live image when performing the fine adjustment. Thus, the examiner is able to position the scan plane at the measurement target smoothly. However, always displaying the assist image 41 and the live image 48 on the same screen, as illustrated in FIG. 5, confuses the examiner as to which image he/she should refer to when moving the probe.

In view of this problem, one aspect of the present invention is an image processing apparatus for generating an assist image that is an image providing guidance in moving an instrument to a target part of a subject, including: a three-dimensional image analyzer determining, as target position information, a three-dimensional position of the target part based on a three-dimensional image including the target part; a position information acquirer acquiring instrument position information indicating a three-dimensional position of the instrument; a display state determiner selecting one display state from at least two display states based on a positional relationship between the target part and the instrument; an assist image generator generating an assist image for the selected display state by using the target position information and the instrument position information; and a display controller performing control for outputting the assist image generated by the assist image generator to a display device.

This achieves displaying, to a user with high visual perceptibility, the image providing guidance in moving the instrument to the target part of the subject.

Further, the at least two display states may include a first display state where the assist image generated by the assist image generator is displayed at a first magnification ratio, and a second display state where the assist image generated by the assist image generator is displayed at a second magnification ratio greater than the first magnification ratio, and the display state determiner may select the first display state when the positional relationship does not fulfill a first predetermined condition, and select the second display state when the positional relationship fulfills the first predetermined condition.

This achieves switching to displaying the assist image in enlarged state when the positional relationship fulfills the first predetermined condition. Accordingly, the assist image is displayed to a user with high visual perceptibility.

Further, the three-dimensional image analyzer may determine, as the target position information, an orientation of the target part based on the three-dimensional image, in addition to determining the three-dimensional position of the target part as the target position information, and the position information acquirer may acquire, as the instrument position information, an orientation of the instrument, in addition to the three-dimensional position of the instrument.

This achieves selecting the display state according to not only the positions of the target part and the instrument, but also the orientations of the target part and the instrument.

Further, the instrument may be a probe in an ultrasound diagnostic device, the probe usable for acquiring an ultrasound image of the subject, the position information acquirer may acquire, as the instrument position information, a scan position and an orientation of the probe, and the assist image generated by the assist image generator may be an image providing guidance in moving the probe to the target part.

This achieves displaying the assist image, which is an image providing guidance in moving the probe to the target part, to a user with high visual perceptibility.

Further, the image processing apparatus may further include a live image acquirer acquiring, from the probe, the ultrasound image of the subject as a live image, and the display controller may output the assist image generated by the assist image generator and the live image to the display device.

This achieves displaying, to a user with high visual perceptibility, both the live image and the assist image.

Further, the at least two display states may include a third display state where on the display device, the assist image generated by the assist image generator is displayed as a main image and the live image is displayed as a sub image, the sub image smaller than the main image, and a fourth display state where on the display device, the live image is displayed as the main image and the assist image generated by the assist image generator is displayed as the sub image, the display state determiner may select the third display state when the positional relationship does not fulfill a second predetermined condition, and select the fourth display state when the positional relationship fulfills the second predetermined condition, and the display controller may output the assist image generated by the assist image generator and the live image to the display device so as to be displayed in the selected display state.

This achieves changing how the live image and the assist image are displayed, so that the live image and the assist image are displayed to a user with high visual perceptibility.

Further, the display controller may output the assist image generated by the assist image generator and the live image to the display device while, based on the selected display state, changing relative sizes at which the assist image generated by the assist image generator and the live image are to be displayed and thereby exchanging the main image and the sub image.

This achieves changing how the live image and the assist image are displayed, so that the live image and the assist image are displayed to a user with high visual perceptibility.

Further, when the third display state is currently selected, the display state determiner may select the display state based on whether the positional relationship fulfills a third predetermined condition, and when the fourth display state is currently selected, the display state determiner may select the display state based on whether the positional relationship fulfills a fourth predetermined condition.

This achieves switching between display states steadily.

Further, the target part may be a blood vessel, and the display state determiner may determine the positional relationship according to whether the live image includes a cross section substantially parallel with a direction in which the blood vessel runs, and select one of the at least two display states based on the positional relationship so determined.

This achieves displaying the assist image, which is an image providing guidance in moving the probe to the target part, to a user with high visual perceptibility.

Further, the image processing apparatus may further include a three-dimensional image generator generating the three-dimensional image from data acquired in advance, and the data acquired in advance may be the ultrasound image, which is obtained by the probe scanning a region including the target part, and the three-dimensional image generator may extract a contour of an organ including the target part from the ultrasound image so as to generate the three-dimensional image, and the three-dimensional image generator may associate a position and an orientation of the three-dimensional image in a three-dimensional space with the scan position and the orientation of the probe acquired by the position information acquirer.

This achieves associating the position and the orientation of the 3D image in the three-dimensional space with the scan position and the orientation of the probe, respectively.

Further, the assist image generator may generate navigation information based on a relative relationship between a current scan position of the probe and the position of the target part, and a relative relationship between a current orientation of the probe and the orientation of the target part, and generate, as the assist image, an image in which the navigation information and a probe image indicating the current scan position and the current orientation of the probe are superimposed on the three-dimensional image.

This achieves displaying the assist image, which is an image providing guidance in moving the probe to the target part, to a user with higher visual perceptibility.

Further, when the fourth display state is selected, the assist image generator may generate a plurality of cross-sectional images each indicating a cross-sectional shape of the target part from one of a plurality of directions, and generate, as the assist image, an image in which a probe image indicating a current scan position and a current orientation of the probe is superimposed on each of the cross-sectional images.

Further, the target part may be a blood vessel, the plurality of cross-sectional images may include two cross-sectional images, one of the two cross-sectional images indicating a cross-sectional shape of the blood vessel from a long axis direction being a direction in which the blood vessel runs, and the other one of the two cross-sectional images indicating a cross-sectional shape of the blood vessel from a short axis direction being substantially perpendicular to the long axis direction, and the assist image generator may generate, as the assist image, an image in which a straight line or a rectangle providing guidance in moving the probe to the target part is superimposed on each of the two cross-sectional images, based on a relative relationship between the current scan position of the probe and the position of the target part and a relative relationship between the current orientation of the probe and the orientation of the target part.

This achieves displaying the assist image, which is an image providing guidance in moving the probe to the target part, to a user with higher visual perceptibility.

Further, the display state determiner may calculate, as the positional relationship, a difference between the position of the target part and the position of the instrument, and a difference between the orientation of the target part and the orientation of the instrument by using the target position information and the instrument position information, and select one of the at least two display states according to the differences so calculated.

Further, the display state determiner may calculate a difference between the position of the target part and the position of the instrument, and a difference between the orientation of the target part and the orientation of the instrument by using the target position information and the instrument position information, and hold the differences so calculated, so as to calculate, as the positional relationship, changes occurring in the differences as time elapses and to select one of the at least two display states according to the changes in the differences so calculated.

This achieves accurate selection of display state.

Further, the target part may be a part of the subject that is a target of surgery, the instrument may be a surgical instrument used in the surgery, and the assist image generated by the assist image generator may be an image providing guidance in moving the surgical instrument to the part of the subject that is the target of surgery.

This achieves allowing a practitioner to confirm the movement of the surgical instrument that he/she has operated, and to adjust with ease the distance of the surgical instrument from the target part and the direction in which he/she performs removal or cutting.

Further, the image processing apparatus may further include a three-dimensional image generator generating the three-dimensional image from data acquired in advance.

This achieves generating a 3D image from data acquired in advance.

Further, the display state determiner may calculate, as the positional relationship, a difference between the position of the target part and the position of the instrument by using the target position information and the instrument position information, and select one of the at least two display states according to the difference so calculated.

Further, the display state determiner may calculate a difference between the position of the target part and the position of the instrument by using the target position information and the instrument position information, and hold the difference so calculated, so as to calculate, as the positional relationship, a change occurring in the difference as time elapses, and select one of the at least two display states according to the change in the difference so calculated.

This achieves accurate selection of display state.

Further, the at least two display states may include two or more display states differing from one another in terms of at least one of a modification ratio and a viewpoint of the assist image, and the display state determiner may select one of the two or more display states based on the positional relationship.

This achieves generating assist images that are in accordance with various forms of display, and displaying assist images to a user with high visual perceptibility.

Such aspects of the present invention, including those that are general and those that are specific, may be realized by a system, a method, an integrated circuit, a computer program, or a computer-readable recording medium such as a CD-ROM, or may be realized by any combination of a system, a method, an integrated circuit, a computer program, or a computer-readable recording medium such as a CD-ROM.

Embodiments of the present invention will be described below with reference to the drawings.

The examples described in the embodiments described below may either be general or specific. The numerical values, the shapes, the materials, the constituent elements, how the constituent elements are arranged in terms of position and are connected with one another, the order of the steps described in the embodiments are mere examples, and thus do not limit the present invention. Further, among the constituent elements described in the embodiments, those not introduced in the independent claims, which represent the present invention in the most general and abstract manner, should be construed as constituent elements that may either be or not be included in the present invention.

### First Embodiment

This embodiment will describe a case where the image processing apparatus pertaining to one aspect of the present invention is implemented as an ultrasound diagnostic apparatus, with reference to the drawings. Note that in the following, a measurement target may be any organ whose image can be captured by ultrasound, and thus, may for example be a blood vessel, the heart, the liver, or the breasts. In the following, description is provided of a case where the measurement target is the carotid artery.

The structure of the ultrasound diagnostic apparatus will be first described.

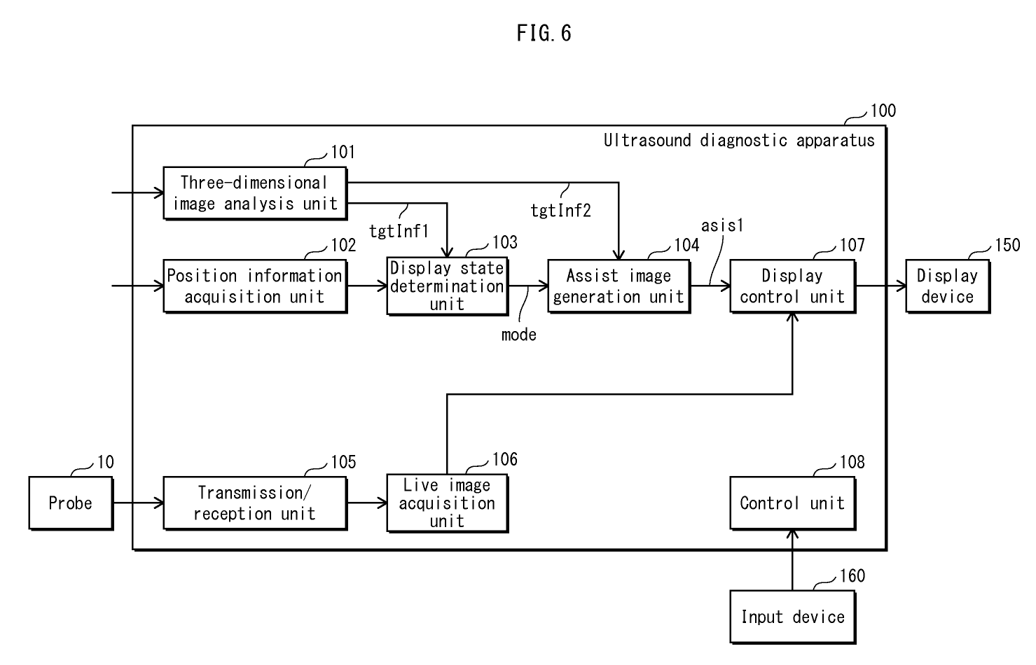

FIG. 6 is a block diagram illustrating the structure of an ultrasound diagnostic apparatus 100 according to the first embodiment.

The ultrasound diagnostic apparatus 100 includes, as shown in FIG. 6, a three-dimensional image analysis unit 101, a position information acquisition unit 102, a display state determination unit 103, an assist image generation unit 104, a transmission/reception unit 105, a live image acquisition unit 106, a display control unit 107, and a control unit 108.

Further, the ultrasound diagnostic apparatus 100 is configured so as to be connectable to a probe 10, a display device 150, and an input device 160.

The probe 10 has a plurality of transducers (not shown) which, for example, are arranged one-dimensionally (hereinafter, transducer array direction). The probe 10 converts a pulse electric signal or a continuous wave electric signal (hereinafter, an electric transmission signal) supplied from the transmission/reception unit 105 into a pulse ultrasound wave or a continuous ultrasound wave. The probe 10 transmits an ultrasound beam composed of a plurality of ultrasound waves generated by the plurality of transducers to the measurement-target organ (i.e., the carotid artery) with the probe 10 in contact with the surface of the subject's skin. In order to acquire a tomographic image of a long-axis cross-section of the carotid artery, the probe 10 should be arranged on the surface of the subject's skin so that the transducer array direction of the probe 10 is along the long-axis direction of the carotid artery. The probe 10 receives a plurality of ultrasound waves reflected off from the subject, and the plurality of transducers convert the reflected ultrasound waves into electric signals (hereinafter, electric reception signal), and supplies the electric reception signals to the transmission/reception unit 105.

Although this embodiment illustrates an example of the probe 10 having a plurality of transducers arrayed one-dimensionally, the probe 10 is not limited to this. For example, the probe 10 may have an array of transducers arranged two-dimensionally, or may be an oscillating ultrasound probe that mechanically oscillates a plurality of transducers arrayed one-dimensionally so as to compose a three-dimensional tomographic image. Different probes may be depending on the measurement to be performed.

Further, the ultrasound probe 10 may be configured to be provided with some of the functions of the transmission/reception unit 105. One example of such a structure is a structure where the probe 10 generates an electric transmission signal based on a control signal (hereinafter, a transmission control signal) which is output from the transmission/reception unit 105 and is for generating the electric transmission signal, and converts this electric transmission signal into an ultrasound wave, and further, generates a reception signal (described later in the present disclosure) based on an electric signal converted from a reflected ultrasound wave that the probe 10 receives.

The display device 150 is a so-called monitor, and displays the output from the display control unit 107 in the form of a displayed screen.

The input device 160 has various input keys, and is used by an operator to make various settings to the ultrasound diagnostic apparatus 100.

FIG. 6 illustrates an example of a structure where the display device 150 and the input device 160 are separate from the ultrasound diagnostic apparatus 100. However, the present invention is not limited to this. For example, a configuration may be made such that the input device 160 operates in accordance with touch panel operations made on the display device 150, and the display device 150 and the input device 160 (and the ultrasound diagnostic apparatus 100) are integrated into a single body.

The three-dimensional image analysis unit 101 analyzes a 3D image that has been acquired in advance through a short-axis scan of the measurement target, and determines position information (target position information) tgtInf1 including a three-dimensional position and an orientation of the measurement target. Further, the three-dimensional image analysis unit 101 outputs the target position information tgtInf 1 so determined to the display state determination unit 103.

The position information acquisition unit 102 acquires position information (instrument position information) indicating a current scan position and a current orientation of the probe 10 by using, for example, a magnetic sensor or an optical camera.

The display state determination unit 103 selects one display state from two display states, based on the positional relationship between the measurement target and the probe 10. Specifically, the display state determination unit 103 selects either a first display state or a second display state, based on the difference between the position of the measurement target and the current scan position, and the difference between the orientation of the measurement target and the orientation of the current scan position. Further, the three-dimensional image analysis unit 101 outputs the display state so selected as mode information mode.

The assist image generation unit 104 acquires, from the three-dimensional image analysis unit 101, assist image generation information tgtInf2 including data of the 3D image and the target position information of the measurement target, and generates an assist image for the display state indicated by the mode information mode. An assist image is an image for providing guidance in moving the probe 10 to the measurement target, and is an image in which information indicating a measurement plane of the measurement target and a position and an orientation of a current scan plane are superimposed on a 3D image. Note that when a magnification ratio, a viewpoint direction, or the like, and not screen structure, is to be switched in the switching of display state, such information related to the magnification ratio, the viewpoint direction, or the like is to be included in the mode information mode. Further, when changing both the magnification ratio and the viewpoint direction in the switching of display state, information related to both the magnification ratio and the viewpoint direction is to be included in the mode information mode.

The transmission/reception unit 105 is connected to the probe 10, and performs a transmission process. The transmission process includes generating a transmission control signal pertaining to ultrasound beam transmission control by the probe 10, and supplying a pulsar electric transmission signal or a continuous wave electric transmission signal generated based on the transmission control signal to the probe 10 Note that the transmission process at least includes generating the transmission control signal and causing the probe 10 to transmit an ultrasound wave (beam).

Meanwhile, the transmission/reception unit 105 also executes a reception process. The reception process includes generating a reception signal by amplifying and A/D converting an electric reception signal received from the probe 10. The transmission/reception unit 105 supplies the reception signal to the live image acquisition unit 106. The reception signal is composed of, for example, a plurality of signals in the transducer array direction and in an ultrasound transmission direction (depth direction), which is perpendicular to the transducer array direction. Each of the signals is a digital signal obtained by A/D-converting an electric signal obtained by converting an amplitude of a corresponding reflected ultrasound wave. The transmission/reception unit 105 repeatedly performs the transmission process and the reception process, to compose a plurality of frames each composed of a plurality of reception signals. The reception process at least includes acquire reception signals based on reflected ultrasound waves.

Here, a frame is one set of reception signals required for composing one tomographic image, a signal that is obtained by processing the set of reception signals to compose tomographic image data, or data corresponding to one tomographic image or a tomographic image that is composed based on the set of reception signals.

The live image acquisition unit 106 generates data of a tomographic image by converting each reception signal in a frame into a luminance signal corresponding to the intensity of the reception signal, and performing coordinate conversion on the luminance signal to convert the luminance signal into coordinates of an orthogonal coordinate system. The live image acquisition unit 106 executes this process successively for each frame, and outputs the tomographic image data so generated to the display control unit 107.

The display control unit 107 causes the display device 150 to display the assist image and a live image, in accordance with the screen structure specified in the mode information mode. In displaying the assist image and the live image, the display control unit 107 respectively uses the assist image generated by the assist image generation unit 104 and an ultrasound live image (tomographic image data) at the current scan position, which is obtained by the live image acquisition unit 106.

The control unit 108 controls the respective units in the ultrasound diagnostic apparatus 100, based on instructions from the input device 160.

The operation of the ultrasound diagnostic apparatus 100 having the above-described structure will be described below.

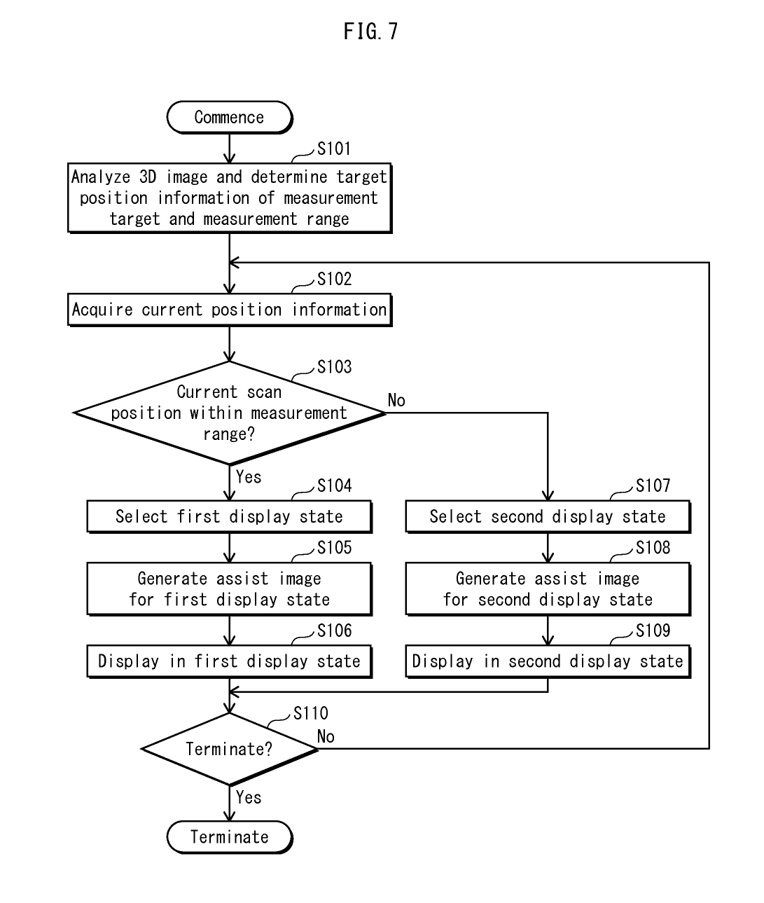

FIG. 7 is a flowchart illustrating the operation of the ultrasound diagnostic apparatus 100 according to the first embodiment.

First, the three-dimensional image analysis unit 101 analyzes the 3D image acquired in advance, and thereby determines the target position information including the position and the orientation of a cross-section that is the measurement target and sets, as a measurement range, a range of positions or orientations differing from the position or the orientation of the measurement target by respective threshold values or less (Step S101).

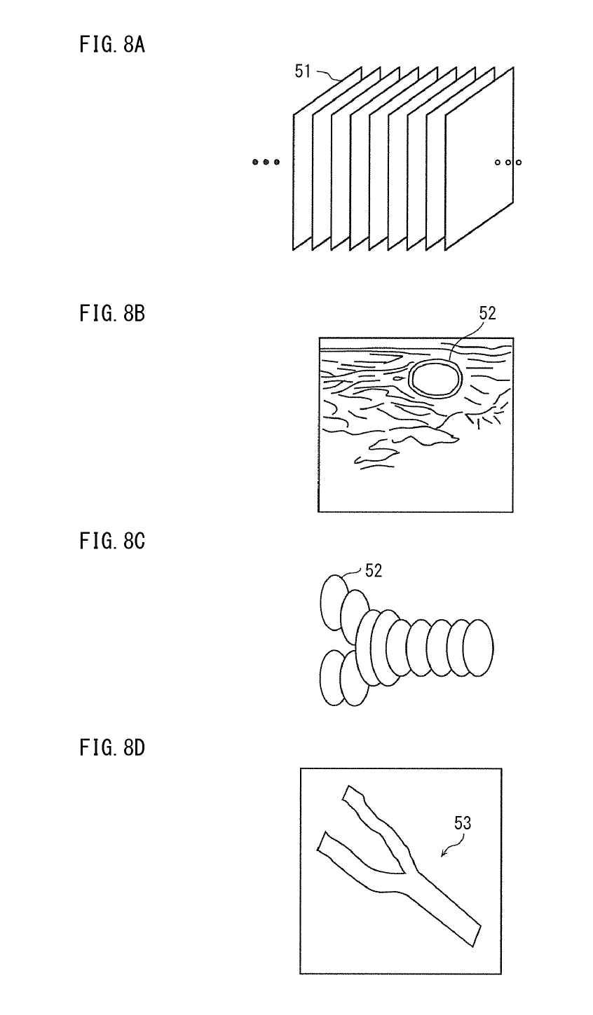

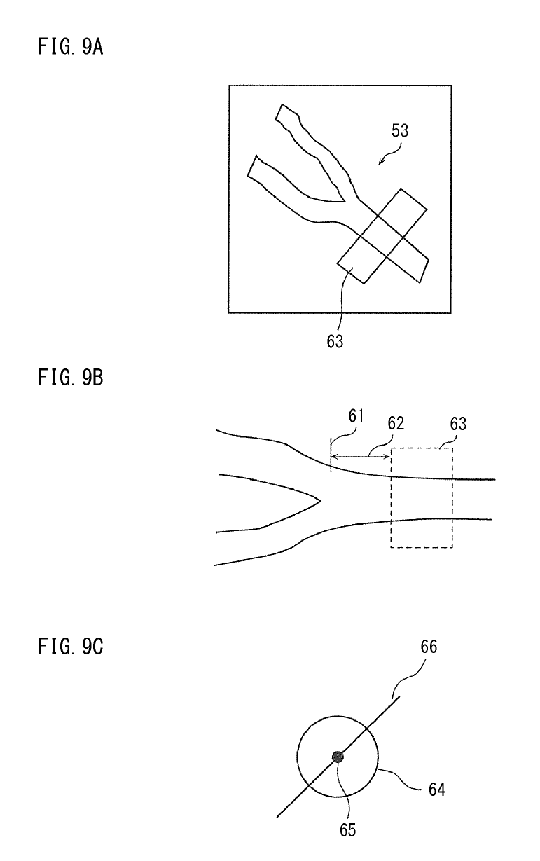

The following describes how the 3D image is generated and how the target position information of a measurement target is determined, with reference to FIGS. 8A to 8D and FIGS. 9A to 9C, respectively. FIGS. 8A to 8D are diagrams illustrating a flow when generating a 3D image by using an ultrasound image.

First, for example, scanning of the entire carotid artery is performed by using the probe 10 to acquire tomographic image data of short-axis images corresponding to a plurality of frames 51, as shown in FIG. 8A, and a vascular contour 52 is extracted from each of the frames 51 of the short-axis images, as shown in FIG. 8B. Then, the vascular contours 52 of the frames 51 are arranged in a three-dimensional space, as shown in FIG. 8C, and further, by generating polygons based on the vertexes of the contours for example, a 3D image 53 of the carotid artery is composed, as shown in FIG. 8D. In the acquisition of each short-axis image, position information (including a position and an orientation) of the scan plane is acquired, and the vascular contours 52 of the frames 51 are arranged in the three-dimensional space based on this position information. The acquisition of the position information is, for example, performed by image-capturing an optical marker attached to the probe 10 by using a camera, and by performing a calculation based on the change in the shape of the optical marker in the images obtained through the image-capturing. Alternatively, the position information may be acquired by using a magnetic sensor, a gyroscope, an acceleration sensor, or the like.

Further, the probe 10 need not be a probe acquiring two-dimensional images, and may be a probe capable of acquiring three-dimensional images without being moved. Examples of such a probe include a mechanically-swinging probe whose scan plane mechanically swings, and a matrix probe in which ultrasound transducers are disposed two-dimensionally on a probe surface.

Further, the 3D image, besides being acquired by using ultrasound, may be acquired through a method such as CT (computer tomography) or MRI (magnetic resonance imaging).

Further, in this embodiment, the 3D image is acquired in advance. However, the present invention is not limited to this, and for example, the ultrasound diagnostic apparatus 100 may be provided with a structure for generating 3D image.

FIG. 9A is a diagram illustrating a position and an orientation of a measurement target in a 3D image. FIG. 9B is a diagram illustrating a position of a measurement target in the long-axis cross-section. FIG. 9C is a diagram illustrating a position of a measurement target in the short-axis cross-section.

The position and the orientation of the measurement target vary according to the purpose of diagnosis of the measurement-target organ. For example, when the measurement-target organ is the carotid artery, typically, a position and an orientation of a measurement target in a 3D image 53 is as shown in FIG. 9A. Therefore, in a long-axis cross-section taken along a direction in which the carotid artery runs, the three-dimensional image analysis unit 101 determines, as a measurement target 63, a portion that is located at a predetermined distance 62 from a measurement reference position 61, as shown in FIG. 9B. The measurement reference position 61 is set based on the shape of the carotid artery.

Further, the three-dimensional image analysis unit 101 determines, as the position of the measurement target 63, a short-axis direction plane corresponding to a plane (hereinafter, maximum active plane) 66 including a line (hereinafter, a center line) 65 connecting centers of contours 64 in the short-axis images of the frames composing the 3D image. The three-dimensional image analysis unit 101 determines the position of the measurement target 63 so that the maximum active plane 66 is a plane including the line connecting the centers of contours around the branch portion of the carotid artery, or a plane tilted by a predetermined angle with respect to such a plane. For example, when the probe can be put in contact along a reference plane passing through the centers of the contours around the branch portion, measurement is conducted at the reference plane. Meanwhile, depending upon the direction in which the carotid artery runs, there are cases where the probe cannot be put in contact along the reference plane. In such a case, it is plausible to select one of two planes that tilted with respect to the reference plane by ±45°. In medical check-ups, it is plausible to conduct measurement of a part that is specified by diagnosis guidelines. Meanwhile, in assessing the effect of plaque treatment, it is important to conduct measurement under the same conditions (position and orientation) every time, as already described above. Therefore, a configuration may be made such that the three-dimensional image analysis unit 101 stores position information of the measurement target acquired through a given diagnostic session, and in the subsequent diagnostic session, three-dimensional image analysis unit 101 determines the measurement target 63 so that measurement can be conducted at the same position and from the same orientation as in the given diagnostic session. Further, the three-dimensional image analysis unit 101 is capable of calculating the thickness of the intima-media complex by extracting the tunica intima boundary and the tunica adventitia boundary from short-axis images and the like acquired in the generation of the 3D image, and further, of detecting a part having a thickness equal to or greater than a threshold value as a plaque. A configuration may be made such that the three-dimensional image analysis unit 101 determines, as the measurement target 63, a long-axis direction cross-section of the plaque so detected where thickness is greatest. Further, in this embodiment, the three-dimensional image analysis unit 101 determines the measurement target 63. Alternatively, an examiner may manually set the measurement target 63.

Subsequently, the position information acquisition unit 102 acquires the position information (instrument position information) indicating the current scan position and the current orientation of the probe 10 (Step S102). Here, the position information acquisition unit 102 acquires this position information by using various sensors, such as a camera and a magnetic sensor, as described above. In an exemplar configuration involving the use of a camera, an optical marker including four markers are attached to the probe 10, and the position information acquisition unit 102 estimates the current scan position and the current orientation of the probe 10 by estimating a position and an orientation of the optical marker based on center coordinates and a size of the area defined by the four markers in the images acquired by the camera.

The display state determination unit 103 determines whether the current scan position is within the measurement range from the measurement target (Step S103). When the current scan position is within the measurement range (Yes in Step S103), the display state determination unit 103 selects the first display state (Step S104). Subsequently, the assist image generation unit 104 generates an assist image for the first display state using the assist image generation information tgtInf2, which includes the data of the 3D image and the target position information of the measurement target (Step S105). Then, the display control unit 107 displays the assist image and the live image, which is an ultrasound image at the current scan position and is acquired by the live image acquisition unit 106, in the first display state on the display device 150 (Step S106).

Meanwhile, when the current scan position is not within the measurement range (No in Step S103), the display state determination unit 103 selects the second display state (Step S107). Subsequently, the assist image generation unit 104 generates an assist image for the second display state using the assist image generation information tgtInf2, which includes the data of the 3D image and the target position information of the measurement target (Step S108). Then, the display control unit 107 displays the assist image and the live image in the second display state on the display device 150 (Step S109).

Following this, a determination is made of whether the process is to be terminated (Step S110), and when the process is not to be terminated (No in Step S110), the process is repeated starting from the acquisition of the current position information (Step S102).

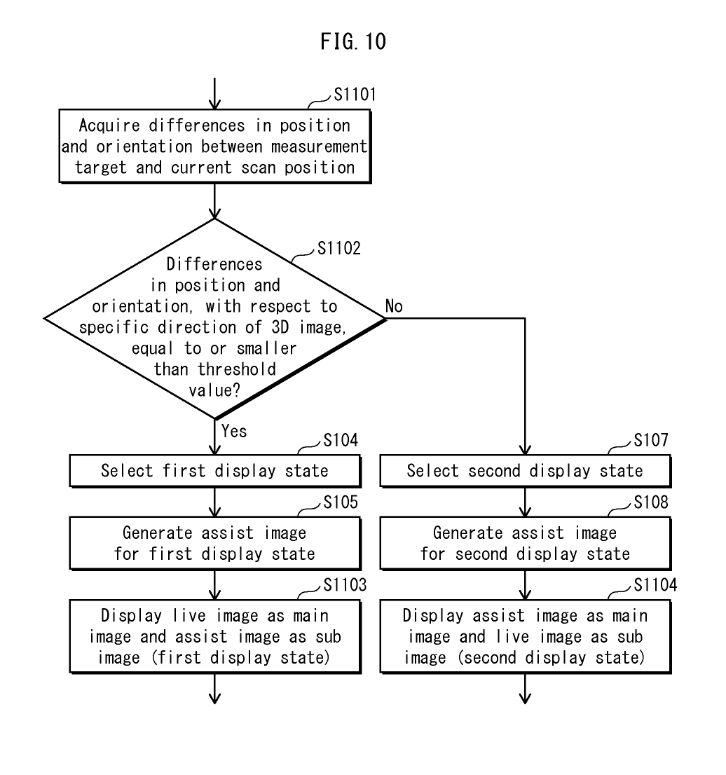

The following describes a specific example of a flow for determining display state illustrated in Steps S103 to S109 in FIG. 7. FIG. 10 is a flowchart illustrating one example of the operation of switching screen display. The flowchart shown in FIG. 10 describes only a part corresponding to Steps S103 to S109 shown in FIG. 7.

The display state determination unit 103 first calculates the difference between the positions of the measurement target and the current scan position and the difference between the orientations of the measurement target and the current scan position (Step S1101). Subsequently, the display state determination unit 103 determines whether the differences, in a specific direction in the 3D image, are equal to or smaller than threshold values (Step S1102).

Here, the specific direction may be the directions of the mutually orthogonal three axes of the three-dimensional coordinate system, or may be a direction set based on the shape of the measurement-target organ. For example, a configuration may be made such that when the measurement target is parallel with the center line of the blood vessel, a determination is made that the differences between the positions and the orientations are equal to or smaller than threshold values when the distance between the center of the measurement target and the center of the scan plane at the current scan position is equal to or smaller than a threshold value and the scan plane at the current scan position is close to parallel with the center line.

When the differences are equal to or smaller than threshold values (Yes in Step S1102), the display state determination unit 103 selects the first display state (Step S104). Subsequently, the assist image generation unit 104 generates an assist image for the first display state using the assist image generation information tgtInf2 (Step S105). Then, the display control unit 107 causes the display device 150 to display in the first display state (fourth display state), where the ultrasound live image is used as a main image and the assist image is used as a sub image (Step S1103).

Meanwhile, when the differences are greater than threshold values (No in Step S1102), the display state determination unit 103 selects the second display state (Step S107). Subsequently, the assist image generation unit 104 generates an assist image for the second display state using the assist image generation information tgtInf2 (Step S108). Then, the display control unit 107 causes the display device 150 to display in the second display state (third display state), where the assist image is used as the main image and the live image is used as the sub image (Step S109).

Here, among the information displayed on a screen of the display device 150, on which ultrasound images are displayed, a main image is an image displayed at the center of the screen or an image that occupies the largest area of the screen, and the sub image is an image displayed at an area other than the area occupied by the main image.

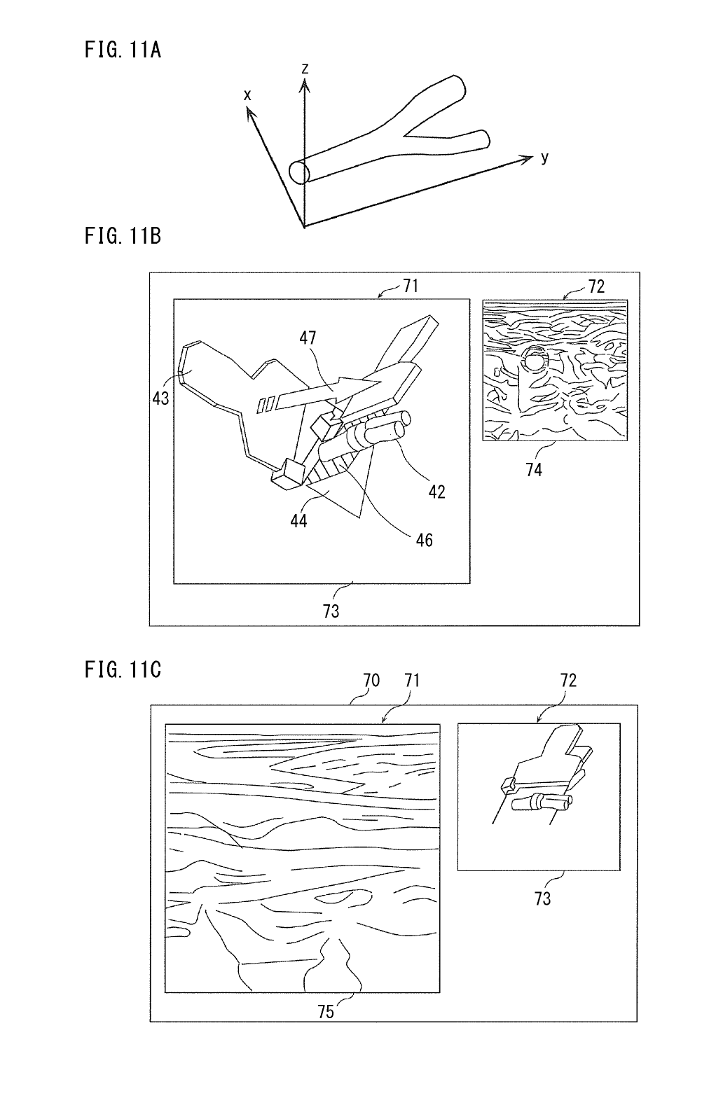

The following describes an example of the switching of display states, conducted when scanning of the measurement target is performed in long-axis images of the carotid artery, with reference to FIG. 11A to FIG. 11C. FIG. 11A is a diagram illustrating one example of the carotid artery (measurement target) in a three-dimensional space. FIG. 11B is a diagram illustrating one example of the second display state. FIG. 11C is a diagram illustrating one example of the first display state.

For measurement of the hypertrophy of the intima-media complex in long-axis images, the probe is first moved close to the measurement target while scanning short-axis images, and then the probe is rotated in order to draw long-axis images. Accordingly, when the short-axis cross-section of the carotid artery is parallel with the x-z plane and the carotid artery runs parallel to the y axis as shown in FIG. 11A, scanning of short-axis images is carried until the probe comes near the position of the measurement target, and then the probe is rotated about the z axis in order to draw long-axis images. Due to this, in Step S1102, a determination is made of whether the current scan position is within the measurement range, or that is, whether the difference between the current scan position and the position of the measurement target in the three-dimensional space is equal to or smaller than a predetermined threshold value and whether the difference between rotation angles about the z axis is equal to or smaller than a predetermined threshold value. This determination enables roughly determining whether long-axis images can be drawn when the probe is rotated about the z axis. Further, by performing this determination, switching between display states is performed when long-axis images can be drawn. The second display state shown in FIG. 11B corresponds to when the current scan position is out of the measurement range, and thus, long-axis images cannot be drawn. In FIG. 11B, an assist image 73 is displayed as a main image 71 and a live image 74 is displayed as sub image 72 on a screen 70 to enable moving the scan position to the target position while mainly referring to the assist image. Meanwhile, the first display state shown in FIG. 11C corresponds to when the current scan position is within the measurement range, and thus, the scan position is near the position of the measurement target. Accordingly, in FIG. 11C, an ultrasound live image 75 is displayed as the main image 71 and the assist image 73 is displayed as the sub image 72 on the screen 70 to enable alignment of positions while mainly with referring to the ultrasound live image 75. The assist image 73 includes a 3D image 42 showing the shape of the organ including the target part, an image 43 showing the current position of the probe 10, an image 44 showing the current scan plane, an image 46 showing a scan plane of the measurement target, an image 45 showing the position to which the probe 10 is to be moved for scanning the measurement target, and an arrow 47 indicating the direction in which the probe 10 is to be moved.

When the screen is switched from the second display state shown in FIG. 11B into the first display state shown in FIG. 11C, the assist image and the live image change positions with one another. However, the switching of display state is not limited to this. For example, by switching the display state to the first display state from the second display state shown in FIG. 11B, the live image 74 may be enlarged to cover a greater area while still being displayed on the right side of the screen. In such a case, the assist image and the live image do not change positions with one another. Further, the switching of the display state is not limited to switching between two patterns. That is, what is displayed on the screen may change continuously through enlarging or reducing respective areas occupied by the live image and the assist image based on the difference between positions and the difference between orientations.

In Step S1102 in FIG. 10, switching of display state is conducted based on whether the difference between positions of the measurement target and the current scan position and the difference between orientations of the measurement target and the current scan position are equal to or smaller than threshold values. Accordingly, when the probe is moved frequently at positions corresponding to differences near the threshold values, switching between display states may be performed restlessly, which results in a decrease in visual perceptibility of the assist image and the live image.

Accordingly, an operation for steadily switching display state will be described below.

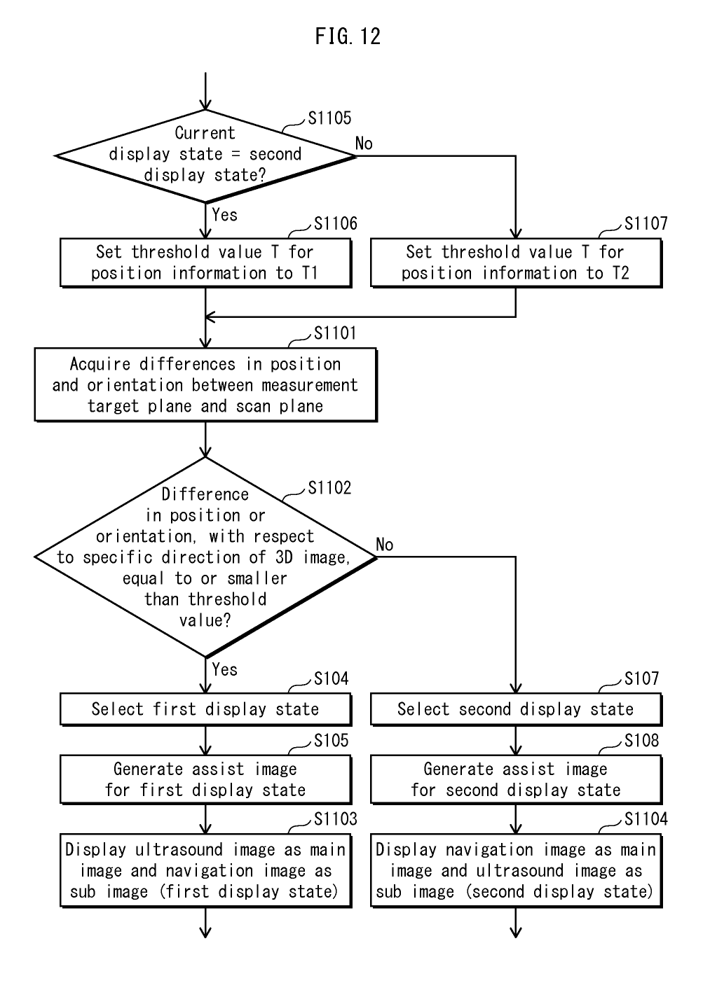

FIG. 12 is a flowchart illustrating an operation for steadily switching display state by introducing hysteresis to the determination of whether or not to switch the display state. Among the steps shown in FIG. 12, Steps S1105 to S1107, which are not included in the flowchart in FIG. 10, will be described below.

The display state determination unit 103 determines whether the current display state is the second display state (Step S1105). When the current display state is the second display state (Yes in Step S1105), the display state determination unit 103 sets T1 as the threshold value to be used for determining whether or not to switch the display state (Step S1106). Here, a different threshold value T1 is set for each of the difference between positions and the difference between orientations. Meanwhile, when the current display state is not the second display state (No in Step S1105), the display state determination unit 103 sets T2 as the threshold value to be used for determining whether or not to switch the display state (Step S1107). Here, T2 is a value differing from T1 set in Step S1106. For example, 8 mm is applied as the threshold value T2 for position when the current display state is the first display state, and 10 mm is applied as the threshold value T1 for the position when the current display state is the second display state. When the current display state is initially the first display state, display state is switched to the second display state when the difference between positions becomes 8 mm or less. Further, since the threshold value applied when the current display state is the first display state is 10 mm, when the difference between positions is equal to or less than 10 mm, the first display state remains to be the current display state. By making such a configuration, even when the probe moves by about 2 mm near where the positional difference is near the threshold value of 8 mm, the display state does not change frequently and remains stable.

Note that the switching performed based on the difference between the position of the measurement target and the current scan position is not limited to switching between different display states, e.g., the main image and the sub image. For example, switching may be performed with respect to parameters affecting the appearance of the assist image itself, such as the viewpoint direction and the magnification ratio of the assist image.

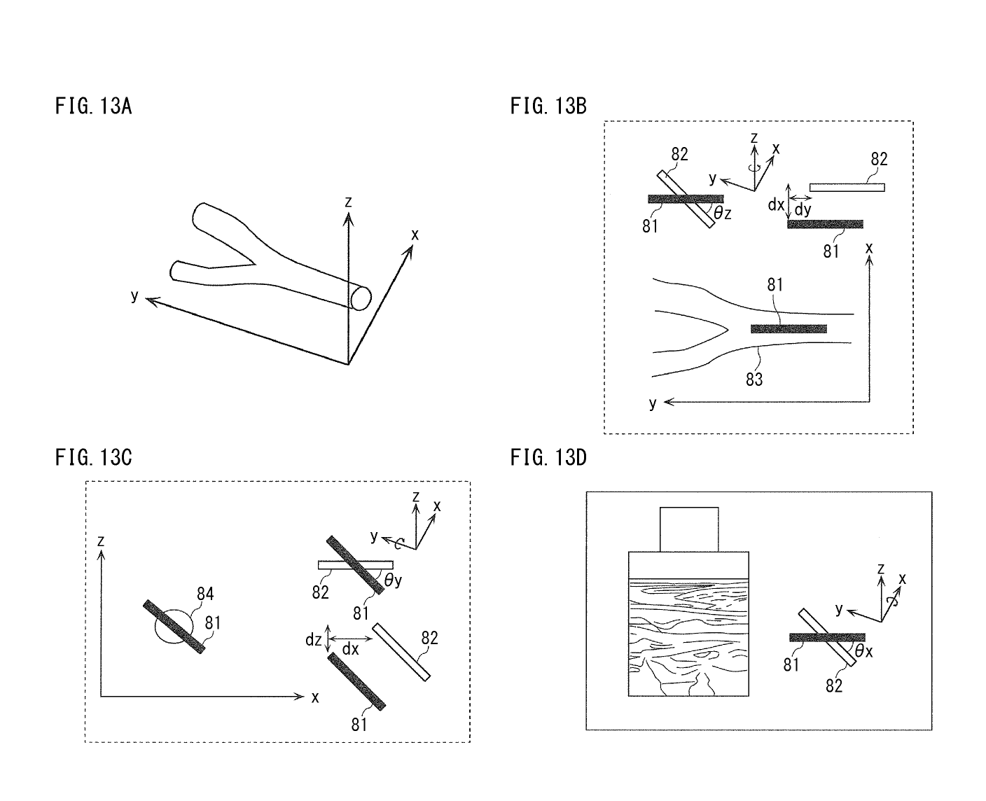

An example of switching the viewpoint direction of the assist image in diagnosis of the carotid artery will be described below with reference to FIG. 13A to FIG. 13D. FIG. 13A is a diagram illustrating one example of the carotid artery (the measurement target) in a three-dimensional space. FIG. 13B is a diagram illustrating one example of the carotid artery in the three-dimensional space viewed in the long-axis direction. FIG. 13C is a diagram illustrating one example of the carotid artery in the three-dimensional space viewed in the short-axis direction. FIG. 13D is a diagram illustrating one example of display performed after the switching, including a combination of a live image from the long-axis direction and an assist image from the short-axis direction.

Here, it is assumed that the carotid artery has a three-dimensional shape as shown in FIG. 13A. In specific, the three-dimensional shape of the carotid artery is such that short-axis cross-sections of the carotid artery are parallel with the x-z plane and the carotid artery runs parallel to the y axis. When measuring the thickness of the intima-media complex of the carotid artery in long-axis images, scanning is first carried out within the measurement range near the measurement target in short-axis images, and then the probe is rotated so that long-axis images are drawn, as described above with reference to FIG. 11A to FIG. 11C. When scanning the short-axis images, the positional relationship between a current scan position 82 and a measurement target 81 is easily perceptible when the viewpoint direction is set to a direction from which the entire long-axis image can be viewed (the z-axis direction in the drawing), as shown in FIG. 13B. Further, when long-axis images have been drawn, it is plausible to set the viewpoint direction to a direction (the y-axis direction in the drawing) from which the scan position and inclination in a short-axis cross-section 84 can be grasped, as shown in FIG. 13C.

Here, as shown in FIG. 13D, by combining a live image with the viewpoint set to the long-axis direction and an assist image with the viewpoint set to the short-axis direction, a form of displaying that facilitates understanding of the positional relationship between the probe 10 and the measurement target can be provided. That is, first, from the inclination of the long-axis image in the live image, information concerning the rotation about the x axis can be acquired. Further, when the direction in which the blood vessel runs (the y-axis direction in the drawing) matches the direction of the scan plane (i.e., when the rotation angles about the z axis in the drawing are the same), a vascular image can be drawn continuously from one end to the other of the screen. However, the larger the misalignment between the angles of rotation about the z axis becomes larger, the smaller the part of the screen in which the vascular image is drawn. Here, it is assumed that the blood vessel meanders slightly. However, at least between the common carotid artery and the branching portion, the blood vessel runs linearly. Thus, this assumption is practical. Therefore, the rotations about the x axis and the z axis, and the position in the y-axis direction can be grasped from the live image. Further, the rotation about the y axis and the positions in the z-axis and z-axis directions can be grasped from the assist image. For this reason, a combination of the live image and the assist image enables all positional relationships to be grasped. Note that the direction in which the blood vessel runs can be determined based on a center line of 3D image.

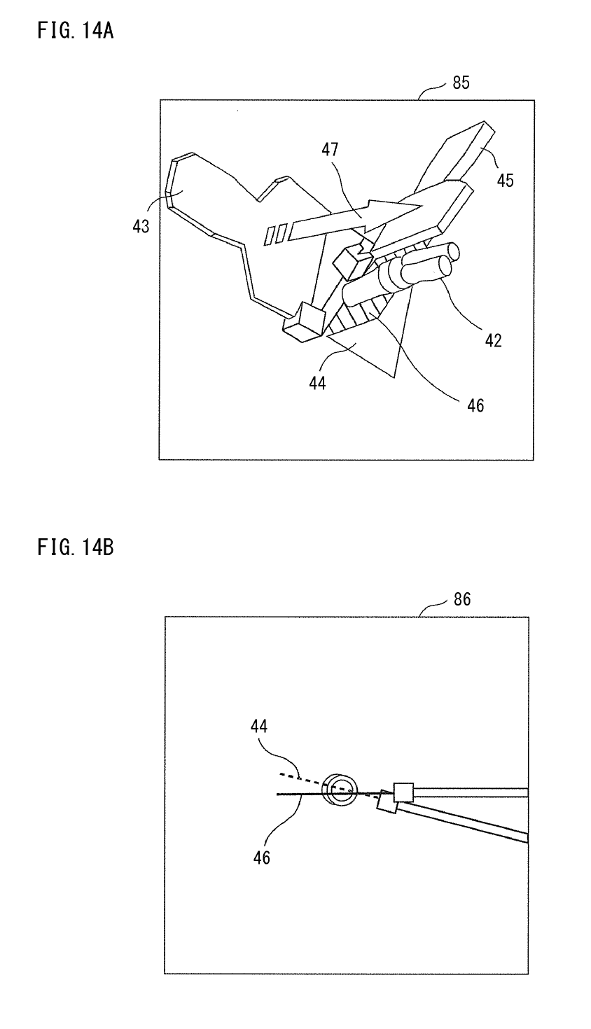

FIG. 14A is a diagram illustrating one example of an assist image before the switching with the viewpoint set to the long-axis direction. FIG. 14B is a diagram illustrating one example of an assist image after the switching with the viewpoint set to the short-axis direction.

When the current scan position is not within the measurement range, an assist image 85 with the viewpoint set to the long-axis direction, as shown in FIG. 14A, is displayed. When the current scan position is within the measurement range, an assist image with the viewpoint set to the short-axis direction, as shown in FIG. 14B, is displayed.

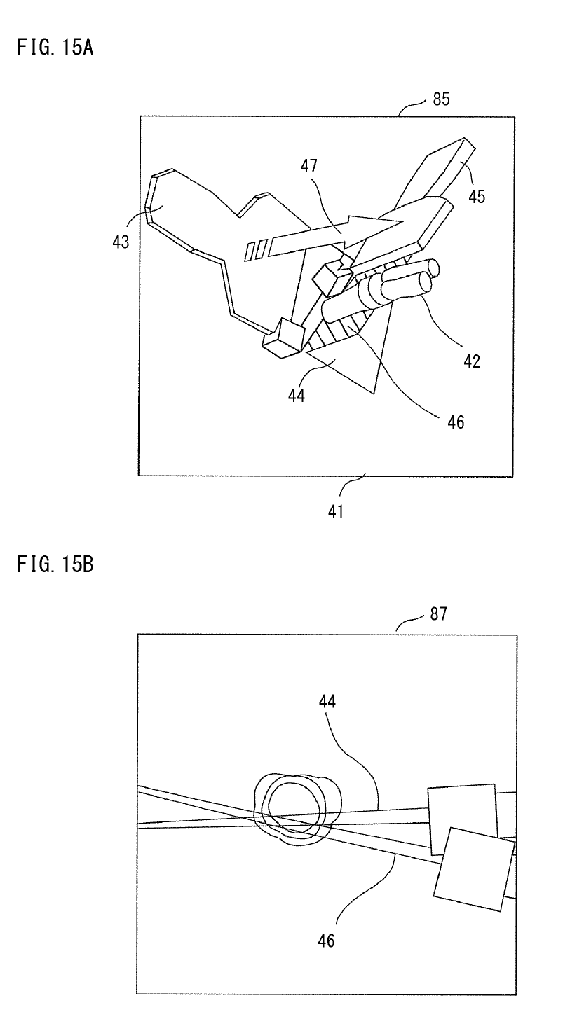

Further, switching of magnification ratio may be performed. FIG. 15A is a diagram illustrating one example of an assist image before the switching of magnification ratio, with the viewpoint set to the long-axis direction. FIG. 15B is a diagram illustrating one example of the assist image after the switching of magnification ratio, with the viewpoint set to the short-axis direction and with increased magnification ratio.

When the current scan position is not within the measurement range and the distance between the scan position and the measurement target is long, the entire image should be viewable. Thus, display is performed with low magnification ratio, as shown in FIG. 15A. When the scan position is within the measurement range and the scan position is to be finely adjusted, display is performed with high magnification ratio being increased as shown in FIG. 15B, so that the region near the measurement target can be viewed precisely.

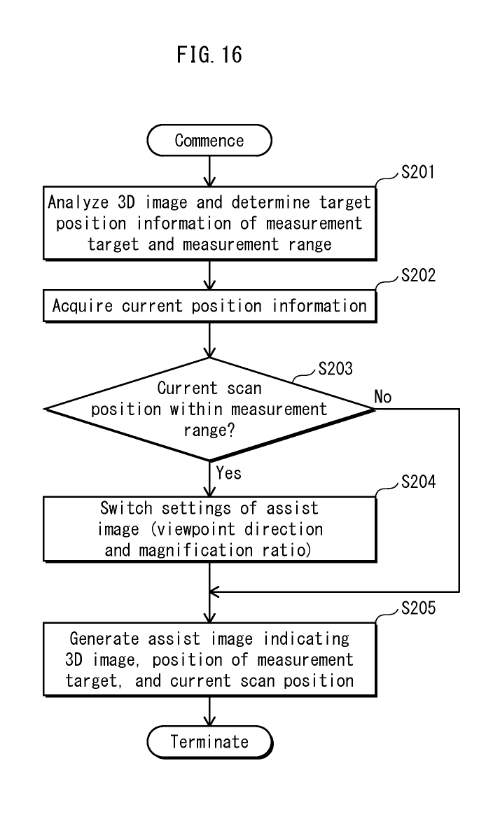

FIG. 16 is a flowchart illustrating one example of an operation for switching settings of the assist image. Steps S201 to S203 are substantially similar to Steps S101 to Step S103 in FIG. 7. The following describes a process in steps S204 and S205.

The assist image generation unit 104 switches settings of parameters of the assist image, such as the viewpoint direction and the magnification ratio (Step S204). Further, the assist image generation unit 104 generates the assist image in which the switching is reflected (Step S205). The switching of the parameters such as the viewpoint direction and the magnification ratio may be used in performed in addition to the switching of screen display.

In the ultrasound diagnostic apparatus 100, the screen display is dynamically switched based on whether the current scan position is within the measurement range of the measurement target. As a result, guidance for moving the probe is provided to the examiner with high visual perceptibility. Further, by making a configuration such that the viewpoint direction of the assist image in the 3D space is changed according to the current scan position and the current orientation, guidance is provided so that the examiner can align the measurement target with the scan position with ease.

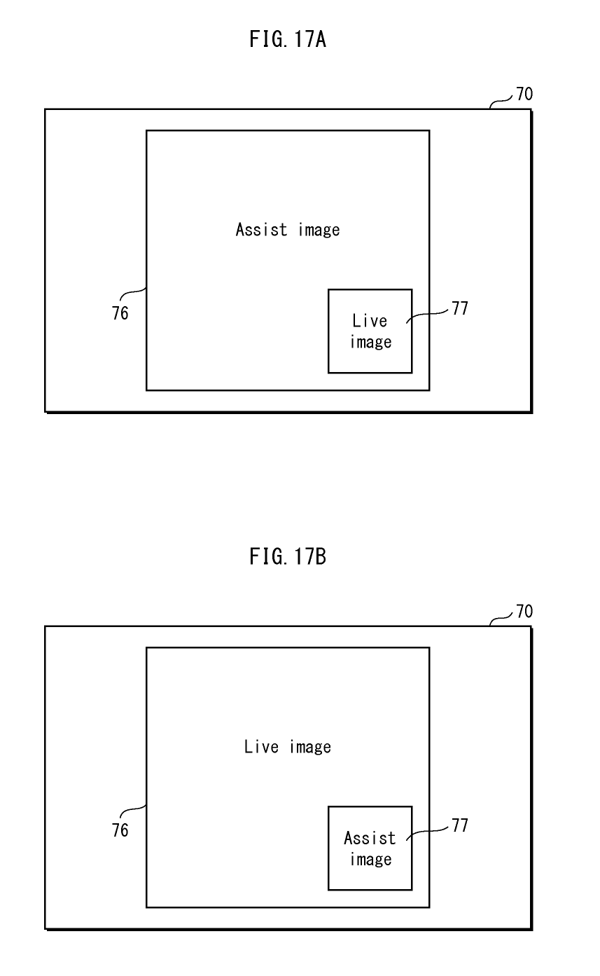

Screen structures other than the screen structures illustrated in FIG. 11B and FIG. 11C may be used. Besides the screen structures shown in FIG. 11B and FIG. 11C, where the main image 71 and the sub image 72 are displayed separately on the screen 70, a screen structure where, as shown in FIG. 17A and FIG. 17B, a main image 76 contains a sub image 77 may be used, for example.

Further, in this embodiment, the display state determination unit 103 selects the first display state or the second display state, based on the difference between the position of the measurement target and the current scan position and the difference between the orientation of the measurement target and the current scan position. However, the present invention is not limited to this. For example, the display state determination unit 103 may select the first display state or the second display state based on the difference between the position of the measurement target and the current scan position. Further, the display state determination unit 103 may retain the difference between the position of the measurement target and the current scan position and the difference between the orientation of the measurement target and the current scan position (or only the difference between the positions), and select the first display state or the second display state, based on a change in the differences taking place as time elapses.

### Second Embodiment

The second embodiment differs from the first embodiment in that the position information acquisition unit 102 of the ultrasound diagnostic apparatus 100 determines whether position information of the probe is acquired. Since the ultrasound diagnostic apparatus 100 in the present embodiment has the same structure as shown in the first embodiment in FIG. 6, the position information acquisition unit 102 will be described by using the same reference symbols.

For example, when acquiring position information by image-capturing an optical marker attached to the probe by using a camera, position information cannot be correctly acquired when the probe leaves the visual field of the camera or the optical marker is hidden by a probe cable or an examiner's hand and is not image-captured by the camera (occlusion). Further, also in a case where, for example, a magnetic sensor is used to acquire the position information, when the probe leaves a magnetic field range or approaches an instrument made of metal or the like that disturbs the magnetic field, the position information of the probe cannot be correctly acquired.

In the second embodiment, the position information acquisition unit 102 determines whether position information of the probe 10 is acquired.

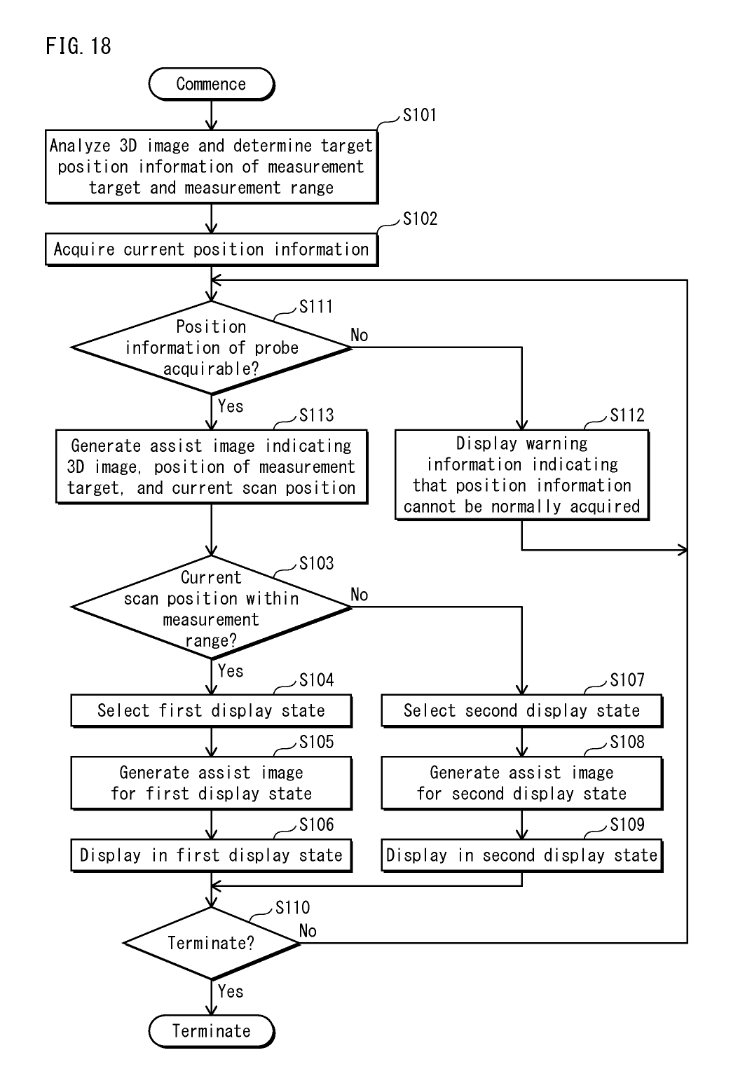

FIG. 18 is a flowchart illustrating the operation of the ultrasound diagnostic apparatus 100 according to the second embodiment. Since steps other than Steps S111 to Step S113 are similar to FIG. 7, description thereof will be omitted.

The position information acquisition unit 102 determines in Step S111 whether the position information of the probe 10 is acquired. When the position information is acquired (Yes in Step S111), the process proceeds to Step S113.

Meanwhile, when the position information is not acquired (No in Step S111), the position information acquisition unit 102 instructs the display control unit 107 to display warning information indicating that the position information is not acquired, and the display control unit 107 displays the warning information on the display device 150 (Step S112).

In this embodiment, when the position information is not acquired, the warning information providing indication that the position information is not acquired is displayed. However, when the position information is acquired in or following Step S103, information providing indication that the position information is acquired may be displayed. Further, in addition to whether or not the position information of the probe can be acquired, display based on reliability of the position information may be performed. For example, when gain, exposure, and/or white balance of the camera is/are not proper, the accuracy in detecting the position of the optical marker in images captured by the camera is low, and accordingly, the reliability of the position information is low. In this case, a numerical value based on reliability or a graphic or the like whose form such as shape, design, and color changes may be displayed in Step S112 or in and following Step S103

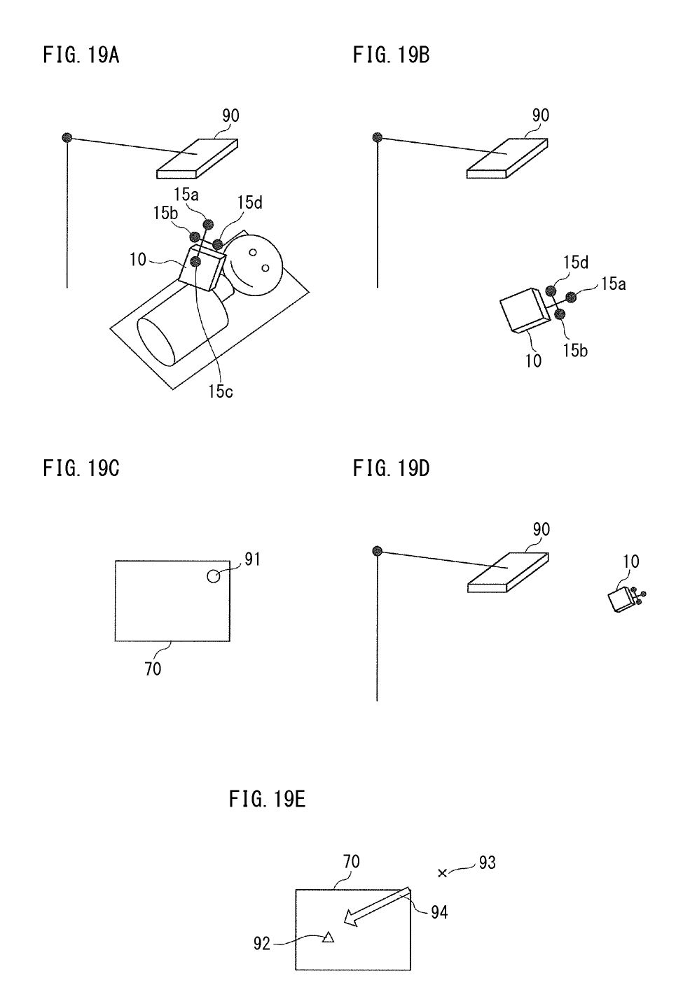

FIG. 19A is a diagram illustrating an example of the structure of a system acquiring the position information of the probe by image-capturing the optical marker attached to the probe by using the camera.

For example, in this system, the optical marker is composed of four markers 15a to 15d as shown in FIG. 19A. The position information acquisition unit 102 estimates the position and the orientation of the optical marker based on the center coordinates and size of the shape composed of the four markers in the images acquired by a camera 90.

FIG. 19B is a diagram illustrating a specific example 1 where the marker 15c cannot be detected due to being hidden by the probe itself, and thus the position information of the probe is not acquired. FIG. 19C is a diagram illustrating a specific example 1 of a screen indicating the warning information.

For example, when the position information is not acquired because the probe 10 is hidden by the marker 15c as shown in FIG. 19B, a red circular symbol 91 indicating that the position information is not acquired is displayed as the warning information on the screen 70 as shown in FIG. 19C. When the position information is acquired, for example, a green circular symbol 91 differing from the red circular symbol, which is one example of the warning information, may be displayed in order to provide indication that the position information is acquired.

FIG. 19D is a diagram illustrating a specific example 2 where the position information is not acquired because the probe 10 is not within the visual field of the camera 90. FIG. 19E is a diagram illustrating a specific example 2 of the screen indicating the warning information.

For example, when the position information is not acquired because the probe 10 is not within the visual field of the camera 90 as shown in FIG. 19D, a x (cross) mark 93 indicating the current position of the probe 10 and an arrow 94 running from the current position of the probe toward a measurement target 92 are displayed on the assist screen, as shown in FIG. 19E. The x mark 93 indicates that the current position of the probe 10 is not within the display range of the assist screen. By displaying such information, notification is provided to the examiner of the direction in which the probe 10 is to be moved to put the probe 10 within the visual field of the camera.

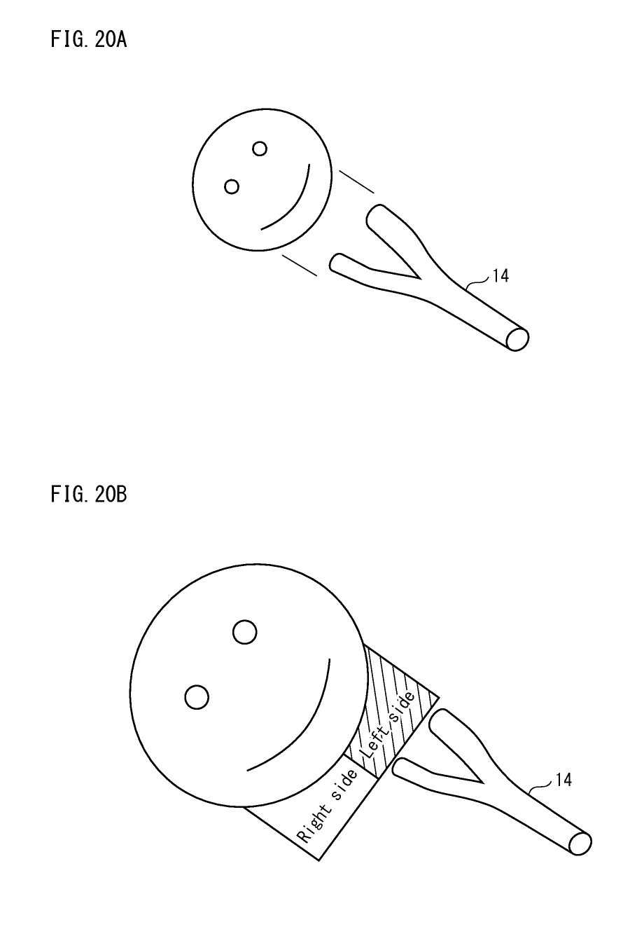

The following describes a modified example of an assist image. FIG. 20A is a diagram illustrating a display example 1 associating the orientation of the 3D image with the posture of the subject. FIG. 20B is a diagram illustrating a display example 2 associating the orientation of the 3D image with the posture of the subject.

For example, information associating the 3D image of the carotid artery with the orientation of the subject's body may be included in the assist image.

For example, an assist image may indicate the orientation of the subject's head as shown in the display example 1 of FIG. 20A, or may indicate whether the 3D image is that of the left or right carotid artery, in addition to the orientation of the subject's head, as shown in the display example 2 of FIG. 20B. The orientation of the head can be determined by detecting, for example, the subject's face or the outline of the subject's head or shoulders from camera images. Alternatively, as the carotid artery branches from one into two in a 3D image of the carotid artery, the direction in which the two blood vessels branching from the carotid artery are present may be determined as the orientation of the head. Alternatively, the orientation of the head may be determined by restricting scan direction in advance such that the scanning direction when performing the short-axis scan for composing the 3D image is the direction from the bottom to the top of the neck.

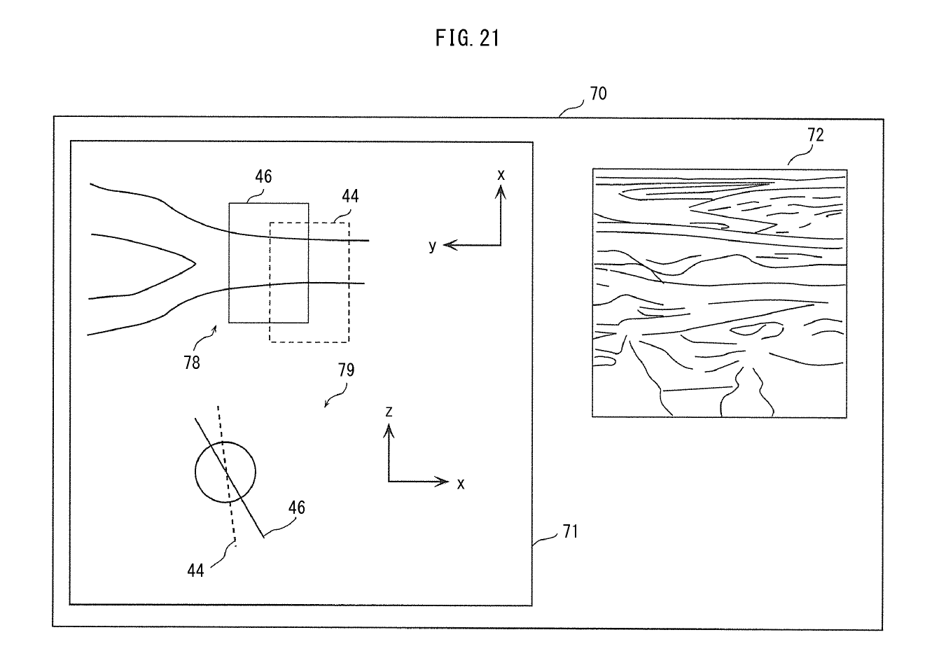

Further, for example, the viewpoint direction need not be switched when switching the main image and the sub image, and the assist image may always include information from a plurality of viewpoint directions. FIG. 21 is a diagram illustrating an example of a structure of a screen in carotid artery diagnosis formed by using an assist image including images (cross-sectional images) from two viewpoint directions, namely from the long-axis direction and the short-axis direction.

In this example, an assist image 71 always has two viewpoint directions, namely the long-axis direction and the short-axis direction. The assist image 71 includes an image 78 from the long-axis direction and an image 79 from the short-axis direction. Further, in the example shown in FIG. 21, the assist image 71 is used in combination with a live image 71, so that information concerning positions and orientations with respect to all three axes, i.e., the x, y, and z axes, is acquired. For this reason, when employing this display, switching of viewpoint direction need not be performed when switching the main image and the sub image.

Further, particularly since a person with skill can draw long-axis images with ease, a configuration may be made such that switching of screen structure is not performed, and a live image is always used as the main image and the assist image is always used as the sub image. Further, a configuration may be made such that information indicating the current scan position is superimposed on the assist image only when the current scan position is within the measurement range. Further, a configuration may be made such that information indicating whether the current scan position is within the measurement range is displayed.

### Third Embodiment

The third embodiment is differs from the first embodiment in that the display state determination unit 103 of the ultrasound diagnostic apparatus 100 switches display state according to whether an ultrasound image includes a long-axis image. Since the ultrasound diagnostic apparatus 100 in the present embodiment has the same structure as shown in the first embodiment in FIG. 6, the display state determination unit 103 will be described by using the same reference symbols.

In the third embodiment, the display state determination unit 103 determines whether an ultrasound image at a current scan position acquired by a live image acquisition unit 106 includes a long-axis image. When the ultrasound image includes a long-axis image, the display state determination unit 103 selects the first display state, where the main image is an ultrasound live image and the sub image is an assist image. Meanwhile, when the ultrasound image does not include a long-axis image, the display state determination unit 103 selects the second display state, where the main image is the assist image and the sub image is the live image.

The tunica intima boundary and the tunica adventitia boundary in a long-axis image of a blood vessel can be extracted based on an ultrasound B-mode image, a color flow image, or a power Doppler image. For example, in order to extract the tunica intima boundary and the tunica adventitia boundary based on a B-mode image, it suffices to search for edges near the boundaries based on brightness values. Further, in order to extract the tunica intima boundary and the tunica adventitia boundary based on a color flow image or a power Doppler image, it suffices to extract vascular contour under the presumption that a blood flow region corresponds to a lumen of the blood vessel. Further, when the direction in which the blood vessel runs and the scan plane of the probe are nearly parallel, an ultrasound image includes a long-axis image from one end to the other. However, the further the scan surface deviates from being parallel to the direction in which the blood vessel runs, the smaller the part of the ultrasound image in which the long-axis image included. Accordingly, when an ultrasound image includes a contour of a long-axis image detected based on a B-mode image or the like with a predetermined length or more, switching of display state can be performed while regarding that the scan plane of the probe is parallel to the direction in which the blood vessel runs. Further, in order to enable the examiner to manually switch display state when determining that a long-axis image is included in an ultrasound image, a configuration may be made of providing a UI (user interface) that facilitates the switching operation such that switching of display state can be performed by a single touch of a button.

The operation of the ultrasound diagnostic apparatus 100 according to the third embodiment will be described below.

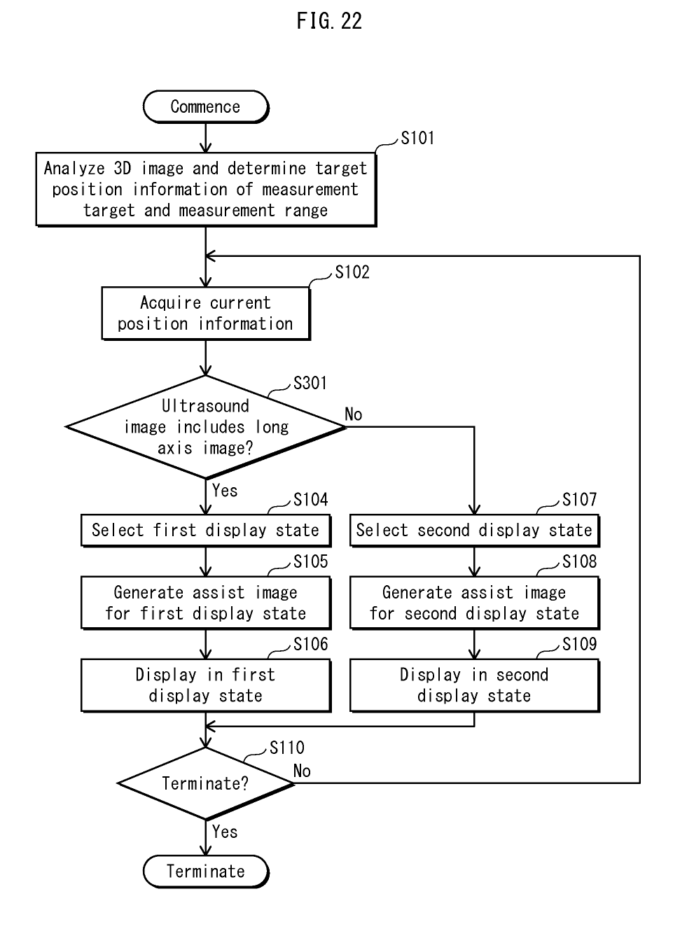

FIG. 22 is a flowchart illustrating the operation of the ultrasound diagnostic apparatus 100 according to the third embodiment. Since steps other than Step S301 are similar to steps in FIG. 7, description thereof will be omitted.

The display state determination unit 103 determines whether a long-axis image is included in the ultrasound image acquired by the live image acquisition unit 106 at the current scan position (Step S301). When a long-axis image is included (Yes in Step S301), the display state determination unit 103 selects the first display state, where the main image is an ultrasound live image and the sub image is an assist image (Step S104). Meanwhile, when a long-axis image is not included (No in Step S301), the display state determination unit 103 selects the second display state, where the main image is an assist image and the sub image is a live image (Step S107).

In this embodiment, the display state on the screen is dynamically switched based on whether a long-axis image is included in an ultrasound image. Accordingly, guidance for moving the probe is provided to the examiner with high visual perceptibility.

The first to third embodiments described above mainly describe operations in the diagnosis of a plaque in the carotid artery. However, assist images are effective not only in plaque diagnosis, but also in Doppler measurement that is important for vascular diagnosis. When applying assist images to Doppler measurement, position information of a sample gate of the Doppler measurement, instead of the position information of a plaque, is determined by the three-dimensional image analysis unit 101 or is set manually. As such, guidance is provided to an examiner so that the examiner can scan a set position of the sample gate. The sample gate can be set at the boundary between the common carotid artery and the carotid sinus, at a predetermined distance from the branching portion of the carotid artery, or near a plaque part. Further, application for observing blood vessels other than the carotid artery, such as the abdominal aorta and the subclavian artery, or for observing tumors in the liver and the breasts is also possible.

### Fourth Embodiment

This embodiment will describe a case where the image processing apparatus according to one aspect of the present invention is applied to an intra-surgery navigation system, with reference to the drawings. An intra-surgery navigation is a system for displaying a positional relationship between a position of a surgical subject part of a patient who is taking a surgery and a surgical instrument. Such an intra-surgery navigation system is used, for example, for improving visual perceptibility of a position of a tumor or a blood vessel, and for improving surgical safety by displaying a position of a surgical instrument with respect to a surgical subject such as a bone or an organ.

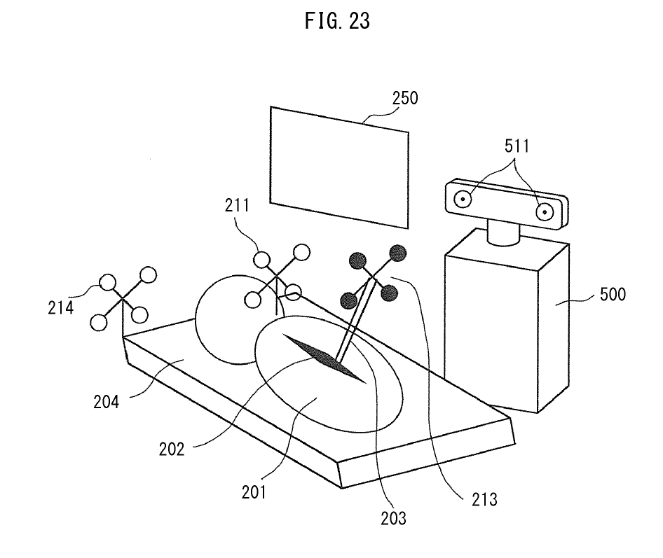

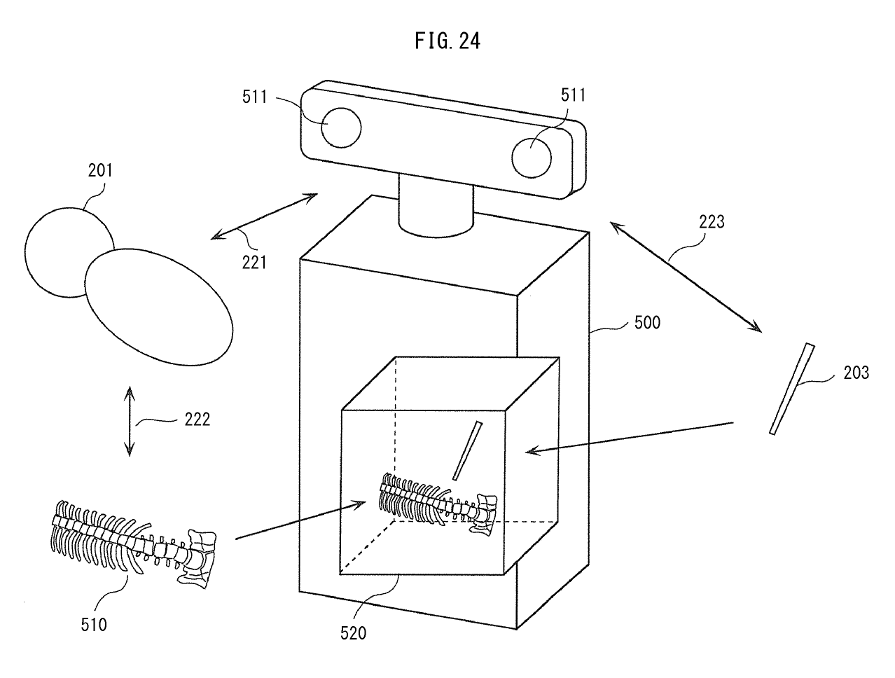

FIG. 23 is a schematic diagram illustrating an example of installation of a intra-surgery navigation system. FIG. 24 is a diagram illustrating an overview of how information is imported to a virtual three-dimensional space.

In a surgical operation, for example, a surgical instrument 203 such as an endoscope may be inserted into an incisional site 202 of a patient 201 (surgical subject), and removal or cutting of a desired part may be performed, as shown in FIG. 23. In such a case, when the desired part cannot be viewed, an intra-surgery navigation system is used to show a practitioner the position of a tip of the surgical instrument 203 in the body of the patient. The intra-surgery navigation system illustrated in FIG. 23 includes an optical marker 213 provided to the surgical instrument 203, a tracking system placed at a bed side of the patient and having an imaging apparatus 511 composed, for example, of one or more CCD camera(s) and an image processing apparatus 500, and a display device (monitor) 250 for displaying navigation information (assist image). The tracking system image-captures the optical marker 213 by using the imaging apparatus 511, and calculates information 223 indicating a spatial position and orientation of the optical marker 213. Further, the tracking system converts the information 223 into information indicating a position and an orientation of the tip of the surgical instrument 203. Further, based on the information so acquired indicating the position and the orientation of the tip of the surgical instrument 203, an object that represents the surgical instrument 203 is arranged in a virtual three-dimensional space 520 that is set in the tracking system.

In recent years, before surgeries, a simulation is conducted to confirm the three-dimensional shape, size, and the like of a surgical subject part of a patient. Further, before surgeries, a region of a surgical-subject patient that is to be removed or cut is determined by using three-dimensional volume data 510 of a surgical target part (target part) acquired by a modality such as a CT, an MRI, a PET, or an ultrasound diagnostic apparatus. Further, when performing intra-surgery navigation, it is necessary to accurately reproduce the actual positional relationship between the patient 201 (surgical subject) and the three-dimensional volume data 510 in a virtual three-dimensional space 520 in the tracking system. As such, it is necessary to measure information 221 indicating the size of the surgical target part, and the position and the orientation of the surgical target part with respect to the tracking system. The actual alignment 222 between the surgical target part and the three-dimensional volume data 510 is carried out before the surgery once the patient is fixed to the bed 204. That is to say, the position, the orientation, and the size of the surgical target part are imported into the tracking system under the condition that the positional relationship between the imaging apparatus 511 and the patient 201 (surgical subject) or the bed 204 to which the patient 201 is fixed does not change. This process is executed by attaching optical markers 214 and 211 to predetermined positions (for example, the bed and a characteristic part of the patient such as a bone) and measuring information indicating the spatial position and orientation of each optical marker by using the tracking system. This is similar to the measurement of information indicating the position and the orientation of the surgical instrument 203.

In such a manner, information indicating the surgical target part and the information indicating the position and the orientation of the surgical instrument are imported into the virtual three-dimensional space in the tracking system.

Setting a given viewpoint position in the virtual three-dimensional space 520 enables generation of an image with which the entirety of the positional relationship between the surgical target part and the surgical instrument can be observed. Further, such an image can be displayed as navigation information (assist image) on the display device 250.

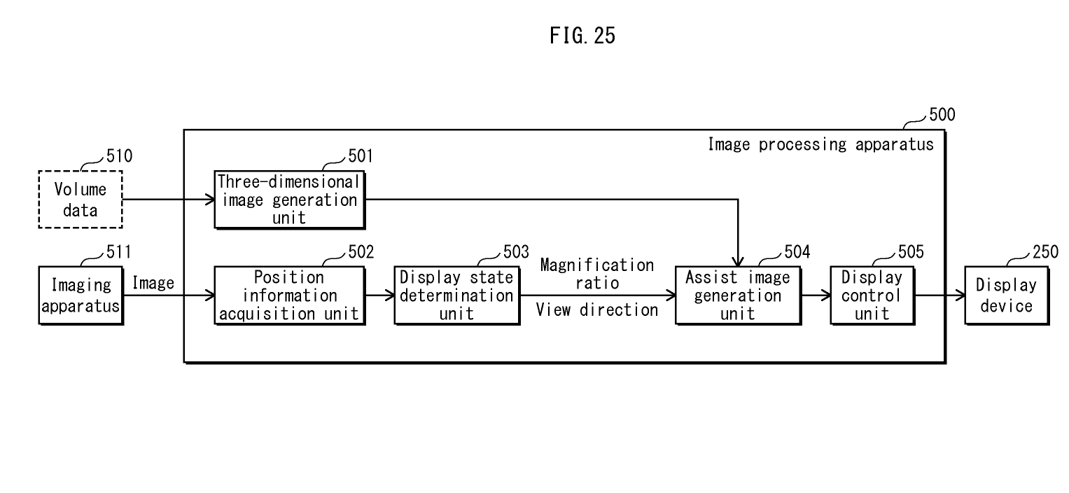

FIG. 25 is a block diagram illustrating the structure of an image processing apparatus 500 according to the fourth embodiment.

The image processing apparatus 500 includes, as shown in FIG. 25, a three-dimensional image generation unit 501, a position information acquisition unit 502, a display state determination unit 503, an assist image generation unit 504, and a display control unit 505. The image processing apparatus 500 is connected to a database storing volume data 510, the imaging apparatus 511, and the display device 250.

The imaging apparatus 511 is an imaging unit such as a CCD camera, and acquires images of the patient (surgical subject) and the surgical instrument. Optical markers appear in the images acquired by the imaging apparatus 511.

The volume data 510 is three-dimensional image data of the surgical target part and is typically acquired by a modality such as a CT or an MRI before surgery. Alternatively, performing navigation while updating the volume data as necessary is possible by using an ultrasound diagnostic apparatus to acquire data in real-time.

The display device 250 is a so-called monitor, and displays the output from the display control unit 505 in the form of a displayed screen.

The three-dimensional image generation unit 501 renders the volume data 510 and generates a 3D image of the surgical target part. Here, the three-dimensional image generation unit 501 may determine a region to be removed or cut, and introduce information of such region and the like to the 3D image.

The position information acquisition unit 502 acquires position information (target position information) including a three-dimensional position and an orientation of the surgical target portion, and position information (instrument position information) indicating a three-dimensional position and an orientation of the surgical instrument. The position information acquisition unit 502 acquires such information based on the images acquired by the imaging apparatus 511, in which the optical markers attached to the surgical instrument, the bed of the surgical subject patient or the surgical subject patient, etc., appear.

The display state determination unit 503 selects one of two display states, based on the positional relationship between the surgical target part (target part) and the surgical instrument. Specifically, the display state determination unit 503 selects either the first display state or the second display state, based on the difference (distance) between the positions of the surgical target part and the surgical instrument. Here, the display state determination unit 503 calculates the distance between the surgical target part and the surgical instrument based on the positions of the surgical target part and the surgical instrument in the virtual three-dimensional space.

The assist image generation unit 504 generates an assist image for the display state selected by the display state determination unit 503.

The display control unit 505 displays the assist image on the display device 250 while controlling the position and the size of the assist image.

The operation of the image processing apparatus 500 having the above structure will be described below.

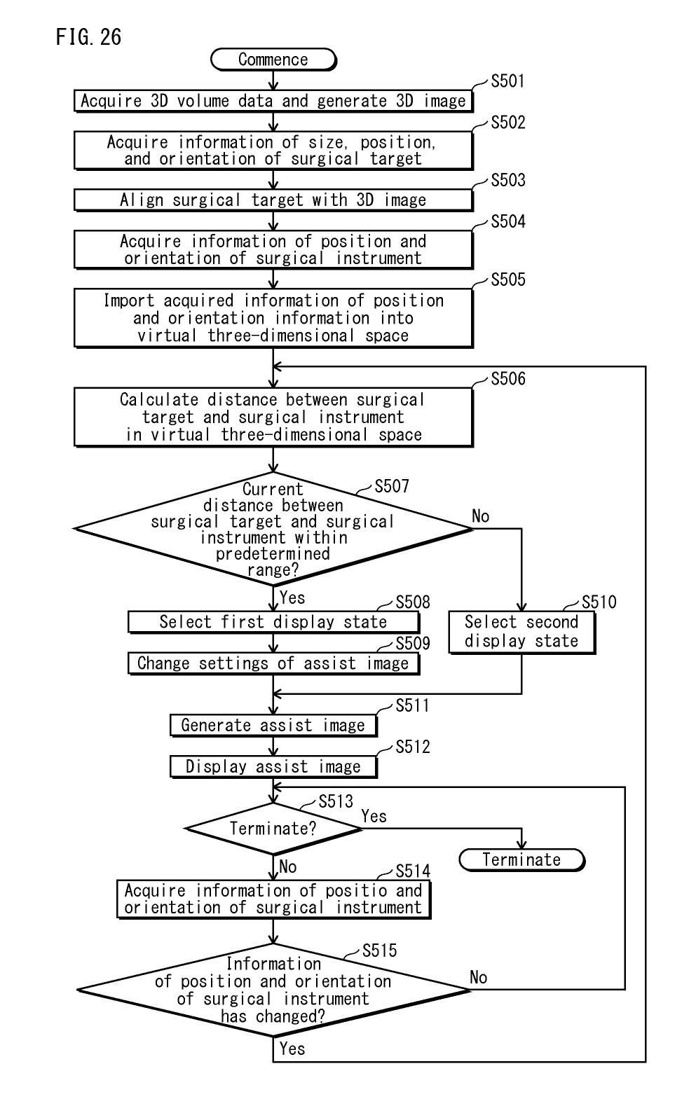

FIG. 26 is a flowchart illustrating the operation of the image processing apparatus 500 according to the fourth embodiment.

The three-dimensional image generation unit 501 acquires pre-acquired 3D volume data that includes the surgical target part of the patient and renders the 3D volume data, so as to generate a 3D image to be included in an assist image (Step S501). Here, the three-dimensional image generation unit 501 may additionally perform a process equivalent to pre-surgery simulation and specify a part to be removed or cut. (Note that typically, the part to be removed or cut is set through a separate process conducted before surgery.)

Subsequently, the position information acquisition unit 502 acquires target position information indicating the three-dimensional position, the orientation, the size, etc., of the surgical target part based on images acquired by the imaging apparatus 511. The imaging apparatus 511 acquires images in an environment where the geometric positional relationship between the imaging apparatus 511 and the bed in the operation room or the surgical subject patient is fixed (Step S502). The three-dimensional image generation unit 501 performs alignment of positions by calibrating the three-dimensional position, the orientation, the size, etc., of the surgical target part with respect to the 3D image (Step S503).

Subsequently, the position information acquisition unit 502 acquires information indicating the position and the orientation of the surgical instrument, based on the images acquired by the imaging apparatus 511. Further, the position information acquisition unit 502 converts the information so that the position and the orientation of the surgical instrument are converted into the position and the orientation of the tip of the surgical instrument, respectively (Step S504).

The three-dimensional image generation unit 501 arranges the surgical target part and the surgical instrument in the virtual three-dimensional space based on information indicating the position and the orientation of the surgical target part, information indicating the position and the orientation of the surgical instrument, information indicating the position and the orientation of the tip of the surgical instrument, and the like (Step S505).

Subsequently, the display state determination unit 503 calculates the distance between the surgical target part and the surgical instrument in the virtual three-dimensional space (Step S506). Then, the display state determination unit 503 determines whether the distance between the surgical target part and the surgical instrument in the virtual three-dimensional space is within a predetermined range (Step S507). When the distance is within the predetermined range (Yes in Step S507), the display state determination unit 103 selects the second display state (Step S508). Further, the display state determination unit 103 changes settings of the assist image, such as the magnification ratio and the view direction (Step S509).

Meanwhile, when the distance is not within the predetermined range (No in Step S507), the display state determination unit 103 selects the first display state (Step S510).

Subsequently, the assist image generation unit 504 generates an assist image for the display state selected by the display state determination unit 503; i.e., the first display state or the second display state (Step S511). Then, the display control unit 505 displays the assist image on the display device 250 (Step S512).

The assist images for the first display state and the second display state will be described.

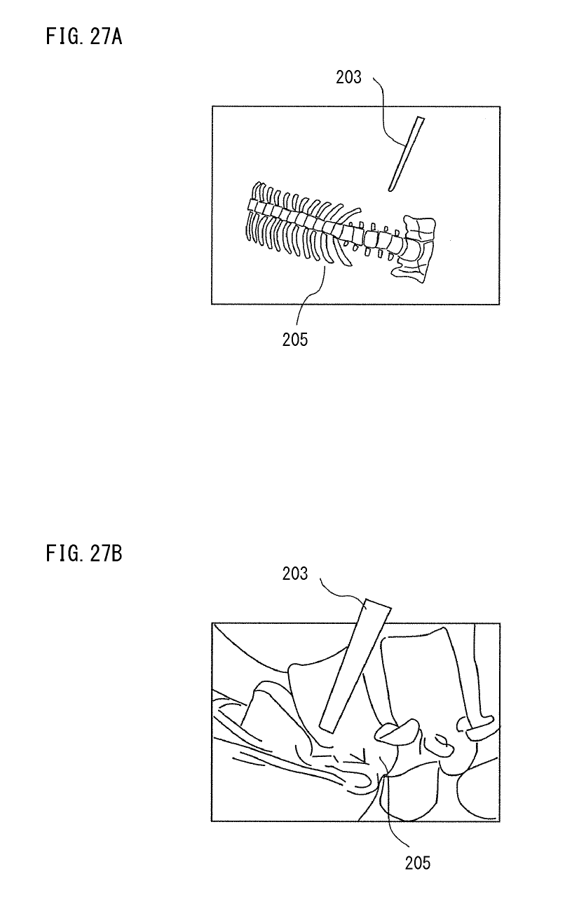

FIG. 27A and FIG. 27B are diagrams illustrating examples of assist images displayed by the image processing apparatus 500. FIG. 27A is a diagram illustrating one example of an assist image for the second display state. FIG. 27B is a diagram illustrating one example of an assist image for the first display state.

The assist image for the first display state is generated when the surgical target part and the surgical instrument are not within the predetermined range (separated by a predetermined distance or more). In the assist image for the first display state, the viewpoint position is set to be distant from the 3D volume data (i.e., a wide field angle is applied in cutting out the image), so that the entirety of the positional relationship between the surgical target part and the surgical instrument can be observed, as shown in FIG. 27A.

Meanwhile, the assist image for the second display state is generated when the surgical target part and the surgical instrument are within the predetermined range (are not separated by the predetermined distance). In the assist image for the second display state, the viewpoint position is set to be close to the 3D volume data (i.e., a narrow field angle is applied in cutting out the image), so that the positional relationship between the surgical target part and the surgical instrument can be observed in more detail and the movement of the surgical instrument can be observed in detail, as shown in FIG. 27B.

Returning to description referring to the flowchart in FIG. 26, subsequently, a determination is made of whether the process is to be terminated (Step S513). When the process is to be terminated (Yes in Step S513), the process is terminated.

Meanwhile, when the process is not to be terminated (No in Step S513), an assist image including information indicating the latest positional relationship between the surgical target part and the surgical instrument is to be generated. As such, the position information acquisition unit 502 acquires information indicating the position and the orientation of the surgical instrument, based on the images acquired by the imaging apparatus 511 (Step S514). Further, a determination is made of whether the position or the orientation of the surgical instrument has changed (Step S515). When the position or the orientation of the surgical instrument has changed (Yes in Step S515), the process starting from Step S506 is repeated.

Meanwhile, when the position or the orientation of the surgical instrument has not changed (No in Step S515), the process starting from Step S513 is repeated. Here, a procedure for updating only the instrument position information of the surgical instrument is described. However, a configuration may be made such that the target position information of the surgical target part is also updated as necessary. When making such a configuration, when the positional relationship between the surgical target part and the surgical instrument has changed, the process starting form Step S506 is repeated.

As such, the instrument position information of the surgical instrument is updated in real time, and accordingly, the assist image displayed on the display device 250 is also updated. Due to this, the practitioner can confirm the movement of the surgical instrument that he/she has manipulated on the display device 250, and thus, is able to adjust with ease the distance between the surgical instrument and the target part, and the direction in which he/she performs the removal or cutting.

In the present embodiment, the assist image generation unit 504 first generates an assist image for the first display state that provides a bird's-eye view, based on the presumption that initially, the surgical target part and the surgical instrument are distant from one another. Subsequently, the display state determination unit 503, when determining that the calculated distance is smaller than a predetermined value, or that is, when determining that the surgical target part and the surgical instrument are very close to each other, changes the settings of the assist image, such as the magnification ratio and the view direction, from the initial settings in Step S509 in order to change the settings of the assist image from those for the first display state to those for the second display state. Further, although not shown in the flowchart of FIG. 26, when the display state is switched back to the first display state after being switching to the second display state, the display state determination unit 503 reverts the settings of the assist image, such as the magnification ratio and the view direction, to the initial settings. Further, the distance calculated by the display state determination unit 503 may be the distance between the center of gravity of the region to be removed or cut in the surgical target part and the tip of the surgical instrument, but is not limited to this.

Further, description has been provided that the three-dimensional image generation unit 501 may perform specification corresponding to pre-surgery simulation and specify the part to be removed or cut in Step S501. Based on this, a configuration may be made such that the result of the simulation (the part to be removed or cut) is superimposed on the 3D image in Step S511. Further, a configuration may be made of additionally providing steps or units for determining whether the surgical instrument has accessed the part to be removed or cut, and updating display by regenerating a 3D image not including the part to be removed or cut when the surgical instrument has accessed the part. This enables the practitioner to grasp the progress of the surgery with more ease.

Further, the present embodiment describes acquiring position information by image-capturing optical markers by using a camera. However, position information may be acquired by using a magnetic sensor, a multi-joint arm, or the like.

Further, the settings of the assist image are switched between two different settings based on information related to distance in Steps S507 to S510. However, the present invention is not limited to this. For example, a modification may be made such that m (where m is a natural number) states for displaying the assist image are prepared in advance, and with an nth one of the display states (where n is a natural number smaller than m) selected, in Step S507, a determination is made of whether or not an absolute value of the difference between a distance at time point t and a distance at time point t−1 is equal to or greater than a predetermined value and whether the difference is positive or negative, and the display state is switched to either the n+1th display state or the n−1th display state based on the determination results. Such a modification achieves an effect where the size in which the part to be removed or cut is enlarged as the surgical instrument approaches the surgical target part. That is, images achieving a smooth transition from FIG. 27A to FIG. 27B can be acquired.

### Fifth Embodiment

By recording, on a recording medium such as a flexible disk, a program for implementing the image processing methods described in the above embodiments, an independent computer system can easily execute processing described in the above embodiments.

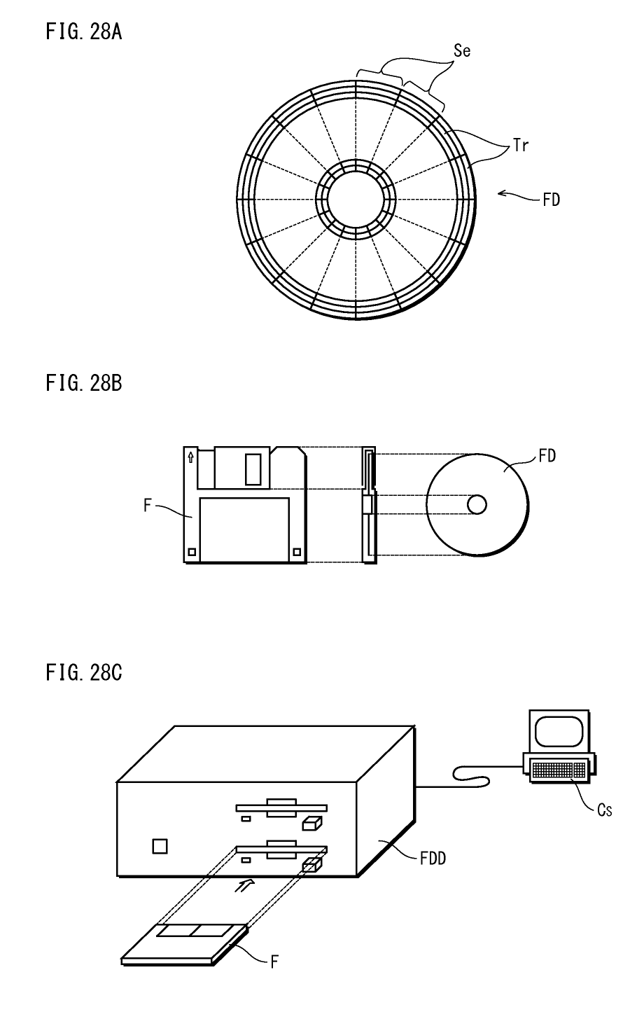

FIGS. 28A through 28C are explanatory diagrams illustrating a case where the image processing methods described in the above embodiments is executed by a computer system using a program recorded on a recording medium such as a flexible disk.

FIG. 28B includes: illustration of an exterior of a floppy disk when seen from a front side, illustration of a cross-sectional structure of the floppy disk, and illustration of an interior of the floppy disk (i.e., the flexible disk). FIG. 28A illustrates an example of a physical format of the flexible disk, which is the main body of a recording medium. The flexible disk FD is housed in a case F. A plurality of tracks Tr are formed on a surface of the flexible disk FD in concentric circles from an outer circumference to an inner circumference of the flexible disk FD. Each track is divided into 16 sectors Se in terms of angle from a center of the flexible disk FD. Therefore, a flexible disk having the above program recorded thereon has, in specific, the above program recorded on a region thereof allocated to the above program.

FIG. 28C illustrates a configuration for recording the program on the flexible disk FD and reproducing the program recorded on the flexible disk FD. When recording a program for implementing ultrasound diagnosis methods on the flexible disk FD, a computer system Cs writes the program to the flexible disk FD via a flexible disk drive. Furthermore, when constructing, in a computer system, the ultrasound diagnosis methods by using the program recorded on the flexible disk, the program is read from the flexible disk via the floppy disk drive and is transmitted to the computer system.

In the above explanation, explanation is provided while taking a flexible disk as an example of a recording medium. However, similar implementation is possible by using an optical disc. Further, recording media are not limited to a flexible disk and an optical disc, and alternatively any media on which the program can be recorded, such as an IC (Integrated Circuit) card or a ROM cassette, can be used for implementation.

Note that functional blocks of the ultrasound diagnostic apparatus illustrated in FIG. 6 and the image processing apparatus illustrated in FIG. 25 are typically implemented by using LSIs, which is one type of an integrated circuit. The implementation of the above-described functional blocks by using LSIs may be performed such that a single LSI chip is used for each individual functional block. Alternatively, the above-described functional blocks may be implemented by using LSIs each including one or more of such functional blocks, or by using LSIs each including a part of each of the functional blocks.

Although referred to here as an LSI, depending on the degree of integration, the terms IC, system LSI, super LSI, or ultra LSI are also used.

In addition, the method for assembling integrated circuits is not limited to the above-described method utilizing LSIs, and a dedicated communication circuit or a general-purpose processor may be used. For example, a dedicated circuit for graphics processing, such as a graphic processing unit (GPU), may be used. A field programmable gate array (FPGA), which is programmable after the LSI is manufactured, or a reconfigurable processor, which allows for reconfiguration of the connection and setting of circuit cells inside the LSI, may alternatively be used.

Furthermore, if technology for forming integrated circuits that replaces LSI were to emerge, owing to advances in semiconductor technology or to another derivative technology, the integration of functional blocks may naturally be accomplished using such technology. The application of biotechnology or the like is possible.

Further, the units of the ultrasound diagnostic apparatus illustrated in FIG. 6 and the image processing apparatus illustrated in FIG. 25 may connect via a network such as the Internet or a local area network (LAN). For example, a configuration may be made such that ultrasound images are read from a server, an accumulation device, etc., located along the network and storing the ultrasound images. Further, a modification may be made such that the adding of functions to the units is performed via a network.

### INDUSTRIAL APPLICABILITY

The image processing apparatus and the image processing method pertaining to the present invention achieve reduction in the time required for positioning a scan position to a target. Thus, the image processing apparatus and the image processing method pertaining to the present invention are expected to improve examination efficiency in screening of arterial sclerosis and the like, and is high usable in the field of medical diagnostic devices.

### REFERENCE SIGNS LIST

10 Probe
30, 100 Ultrasound diagnostic apparatus
31, 100 Three-dimensional image analysis unit
32, 102, 502 Position information acquisition unit
33, 104, 504 Assist image generation unit
34, 106 Live image generation unit
35, 107, 505 Display control unit
103, 503 Display state determination unit
105 Transmission/reception unit
108 Control unit
150, 250 Display device
160 Input device
500 Image processing apparatus
501 Three-dimensional image generation unit
510 Volume data
511 Imaging apparatus

1. An image processing apparatus for generating an assist image that is an image providing guidance in moving an instrument to a target part of a subject, comprising:
a three-dimensional image analyzer determining, as target position information, a three-dimensional position of the target part based on a three-dimensional image including the target part;
a position information acquirer acquiring instrument position information indicating a three-dimensional position of the instrument;
a display state determiner selecting one display state from at least two display states based on a positional relationship between the target part and the instrument;
an assist image generator generating an assist image for the selected display state by using the target position information and the instrument position information; and
a display controller performing control for outputting the assist image generated by the assist image generator to a display device.

2. The image processing apparatus according to claim 1, wherein:
the at least two display states include:
a first display state where the assist image generated by the assist image generator is displayed at a first magnification ratio, and
a second display state where the assist image generated by the assist image generator is displayed at a second magnification ratio greater than the first magnification ratio, and
the display state determiner selects the first display state when the positional relationship does not fulfill a first predetermined condition, and selects the second display state when the positional relationship fulfills the first predetermined condition.

3. The image processing apparatus according to claim 1, wherein:
the three-dimensional image analyzer determines, as the target position information, an orientation of the target part based on the three-dimensional image, in addition to determining the three-dimensional position of the target part as the target position information, and
the position information acquirer acquires, as the instrument position information, an orientation of the instrument, in addition to the three-dimensional position of the instrument.

4. The image processing apparatus according to claim 3, wherein:
the instrument is a probe in an ultrasound diagnostic device, the probe usable for acquiring an ultrasound image of the subject,
the position information acquirer acquires, as the instrument position information, a scan position and an orientation of the probe, and
the assist image generated by the assist image generator is an image providing guidance in moving the probe to the target part.

5. The image processing apparatus according to claim 4, further comprising:
a live image acquirer acquiring, from the probe, the ultrasound image of the subject as a live image, wherein the display controller outputs the assist image generated by the assist image generator and the live image to the display device.

6. The image processing apparatus according to claim 5, wherein:
the at least two display states include:
a third display state where on the display device, the assist image generated by the assist image generator is displayed as a main image and the live image is displayed as a sub image, the sub image smaller than the main image, and
a fourth display state where on the display device, the live image is displayed as the main image and the assist image generated by the assist image generator is displayed as the sub image,
the display state determiner selects the third display state when the positional relationship does not fulfill a second predetermined condition, and selects the fourth display state when the positional relationship fulfills the second predetermined condition, and
the display controller outputs the assist image generated by the assist image generator and the live image to the display device so as to be displayed in the selected display state.

7. The image processing apparatus according to claim 6, wherein the display controller outputs the assist image generated by the assist image generator and the live image to the display device while, based on the selected display state, changing relative sizes at which the assist image generated by the assist image generator and the live image are to be displayed and thereby exchanging the main image and the sub image.

8. The image processing apparatus according to claim 6, wherein when the third display state is currently selected, the display state determiner selects the display state based on whether the positional relationship fulfills a third predetermined condition, and when the fourth display state is currently selected, the display state determiner selects the display state based on whether the positional relationship fulfills a fourth predetermined condition.

9. The image processing apparatus according to claim 5, wherein:
the target part is a blood vessel, and
the display state determiner determines the positional relationship according to whether the live image includes a cross section substantially parallel with a direction in which the blood vessel runs, and selects one of the at least two display states based on the positional relationship so determined.

10. The image processing apparatus according to claim 4, further comprising:
a three-dimensional image generator generating the three-dimensional image from data acquired in advance, wherein:
the data acquired in advance is the ultrasound image, which is obtained by the probe scanning a region including the target part, and the three-dimensional image generator extracts a contour of an organ including the target part from the ultrasound image so as to generate the three-dimensional image, and
the three-dimensional image generator associates a position and an orientation of the three-dimensional image in a three-dimensional space with the scan position and the orientation of the probe acquired by the position information acquirer.

11. The image processing apparatus according to claim 4, wherein
the assist image generator generates navigation information based on a relative relationship between a current scan position of the probe and the position of the target part, and a relative relationship between a current orientation of the probe and the orientation of the target part, and generates, as the assist image, an image in which the navigation information and a probe image indicating the current scan position and the current orientation of the probe are superimposed on the three-dimensional image.

12. The image processing apparatus according to claim 6, wherein
when the fourth display state is selected, the assist image generator generates a plurality of cross-sectional images each indicating a cross-sectional shape of the target part from one of a plurality of directions, and generates, as the assist image, an image in which a probe image indicating a current scan position and a current orientation of the probe is superimposed on each of the cross-sectional images.

13. The image processing apparatus according to claim 12, wherein:
the target part is a blood vessel,
the plurality of cross-sectional images includes two cross-sectional images, one of the two cross-sectional images indicating a cross-sectional shape of the blood vessel from a long axis direction being a direction in which the blood vessel runs, and the other one of the two cross-sectional images indicating a cross-sectional shape of the blood vessel from a short axis direction being substantially perpendicular to the long axis direction, and
the assist image generator generates, as the assist image, an image in which a straight line or a rectangle providing guidance in moving the probe to the target part is superimposed on each of the two cross-sectional images, based on a relative relationship between the current scan position of the probe and the position of the target part and a relative relationship between the current orientation of the probe and the orientation of the target part.

14. The image processing apparatus according to claim 3, wherein the display state determiner calculates, as the positional relationship, a difference between the position of the target part and the position of the instrument, and a difference between the orientation of the target part and the orientation of the instrument by using the target position information and the instrument position information, and selects one of the at least two display states according to the differences so calculated.

15. The image processing apparatus according to claim 3, wherein the display state determiner calculates a difference between the position of the target part and the position of the instrument, and a difference between the orientation of the target part and the orientation of the instrument by using the target position information and the instrument position information, and holds the differences so calculated, so as to calculate, as the positional relationship, changes occurring in the differences as time elapses and to select one of the at least two display states according to the changes in the differences so calculated.

16. The image processing apparatus according to claim 1, wherein:
the target part is a part of the subject that is a target of surgery,
the instrument is a surgical instrument used in the surgery, and
the assist image generated by the assist image generator is an image providing guidance in moving the surgical instrument to the part of the subject that is the target of surgery.

17. The image processing apparatus according to claim 16, further comprising:
a three-dimensional image generator generating the three-dimensional image from data acquired in advance.

18. The image processing apparatus according to claim 1, wherein the display state determiner calculates, as the positional relationship, a difference between the position of the target part and the position of the instrument by using the target position information and the instrument position information, and selects one of the at least two display states according to the difference so calculated.

19. The image processing apparatus according to claim 1, wherein the display state determiner calculates a difference between the position of the target part and the position of the instrument by using the target position information and the instrument position information, and holds the difference so calculated, so as to calculate, as the positional relationship, a change occurring in the difference as time elapses, and select one of the at least two display states according to the change in the difference so calculated.

20. The image processing apparatus according to claim 1, wherein:
the at least two display states include two or more display states differing from one another in terms of at least one of a modification ratio and a viewpoint of the assist image, and
the display state determiner selects one of the two or more display states based on the positional relationship.

21. An image processing method for generating an assist image that is an image providing guidance in moving an instrument to a target part of a subject, comprising:
determining, as target position information, a three-dimensional position of the target part based on a three-dimensional image including the target part;
acquiring instrument position information indicating a three-dimensional position of the instrument;
selecting one display state from at least two display states based on a positional relationship between the target part and the instrument;
generating an assist image for the selected display state by using the target position information and the instrument position information; and
performing control for outputting the assist image generated by the assist image generator to a display device.

22. A non-transitory computer-readable recording medium having recorded thereon a program for generating an assist image that is an image providing guidance in moving an instrument to a target part of a subject, the program causing a computer to execute:
determining, as target position information, of a three-dimensional position of the target part based on a three-dimensional image including the target part;
acquiring of instrument position information indicating a three-dimensional position of the instrument;
selecting of one display state from at least two display states based on a positional relationship between the target part and the instrument;
generating of an assist image for the selected display state by using the target position information and the instrument position information; and
performing of control for outputting the assist image generated by the assist image generator to a display device.
